(19) United States
(12) Patent Application Publication
(10) Pub. No.: US 2023/0120498 A1
(43) Pub. Date: Apr. 20, 2023

(54) COVER TYPE ELECTRONIC DEVICE AND METHOD FOR MEASURING BIOMETRIC DATA THEREOF

(71) Applicant: SAMSUNG ELECTRONICS CO., LTD., Suwon-si (KR)

(72) Inventors: Joongwoo AHN, Suwon-si (KR); Seungju KIM, Suwon-si (KR); Suho LEE, Suwon-si (KR); Seunghyun LEE, Suwon-si (KR); Jeehoon LEE, Suwon-si (KR); Hyungjoon LIM, Suwon-si (KR); Shinhee CHO, Suwon-si (KR); Jinho CHOI, Suwon-si (KR); Hojin HWANG, Suwon-si (KR)

(21) Appl. No.: 18/069,848

(22) Filed: Dec. 21, 2022

Related U.S. Application Data
(63) Continuation of application No. PCT/KR2022/005696, filed on Apr. 21, 2022.

(30) Foreign Application Priority Data
May 20, 2021 (KR) 10-2021-0064938

(51) Int. Cl. A61B 5/053; A61B 5/282

(52) U.S. Cl. CPC A61B 5/053; A61B 5/282

(57) ABSTRACT

A cover-type electronic device includes a cover-type housing, an electrode set including a plurality of electrodes disposed on an exterior surface of the housing, and a printed circuit board electrically connected to the plurality of electrodes. A driving circuit in the printed circuit board is configured to acquire sensing information, and identify a user's gripping posture, which matches one of a plurality of gripping postures for measurement of designated biometric data, based on the sensing information. The driving circuit identifies information corresponding to a contact impedance difference between both hands of the user from a biosignal detected through the electrode set, and switches a connection state of the electrode set, based on the corresponding information such that the user's biometric data is measured in the switched state.

### CROSS-REFERENCE TO RELATED APPLICATIONS

This application is a bypass continuation of International Application No. PCT/KR2022/005696, filed Apr. 21, 2022, which claims priority to Korean Patent Application No. 10-2021-0064938, filed May 20, 2021, the disclosures of which are herein incorporated by reference in their entirety.

### BACKGROUND

### 1. Field

Various embodiments disclosed herein relate to a cover-type electronic device and a method for measuring biometric data thereby.

### 2. Description of Related Art

In line with increasing interests in health and medicine, biometric signal measuring functions have been widely adopted by portable or mobile electronic devices (for example, smartphones, wearable devices). An electronic device may contact the user's body and measure the user's biometric signal. For example, the electronic device may measure biometric signals such as the user's electrocardiography (ECG), heart rate, electrodermal activity (EDA), and/or bioelectrical impedance.

For example, the ECG is a test of the electrical activity of the heart, and may be measured by attaching an electrode to skin, connecting the electrode to external equipment, and recording biometric signals detected through the electrode. 3 During an ECG test, twelve ECG leads regarding transverse directions of the heart (voltage difference tracks between two electrodes) may be measured and observed. The result may be used to diagnose various heart diseases such as myocardial infarction, arrhythmia, and hyperpotassemia.

A biometric signal test scheme using a specialized device (for example, medical special test device) requires expensive devices and dedicated personnel, and may not be easily accessed by people with negligible symptoms or no symptoms.

There has recently been increasing use of health care services using versatile electronic devices (for example, smartphones, wearable devices). For example, according to a currently available a health care service, only one of twelve ECG leads (for example, lead I, left hand: + electrode, right hand: − electrode) is measured to screen abnormal activities of the heart or to recommend a hospital visit.

If such a health care service is provided by a versatile electronic device (for example, smartphone, wearable device) instead of a specialized device (for example, medical special test device), the high degree of portability or mobility may increase the service utilization or satisfaction, and may make use of the health care service during daily life convenient.

### SUMMARY

For a health care service, a specialized portable measurement device (for example, ECG measurement device) may be connected to a versatile electronic device (for example, smartphone, wearable device) and used. In this case, there user may be inconvenienced by the fact that an additional measurement device needs to be carried in addition to the versatile electronic device or needs to wear the same in a correct manner.

Existing measurement devices have fixed electrode structures and, if users do not take the correct measurement posture, normal measurement may be difficult (e.g., impossible in some instances) to attain, or the measurement result may have degraded reliability. For example, when measuring ECG lead I, a plug electrode may be attached to the user's left hand, and a minus electrode to the right hand, thereby measuring the voltage difference between both arms. If the user measures in the opposite direction to the determined direction or measures in a different posture, the ECG waveform may be inverted, or the measurement may fail.

If a portable or mobile versatile electronic device (for example, smartphone, wearable device) has additional electrodes for biometric data measurement, the design flexibility may be limited by the limited area. It may be difficult to implement an electric separation structure between the electrodes or between the device body and the electrodes, or the unique performance of the device (for example, antenna performance) may be adversely affected.

If a metal (dry) metal is used for use convenience in connection with a portable measurement device or a versatile electronic device, measurement may be impossible in some instances or difficult depending on the user's physical condition. For example, if the user has dry skin, has many dead skin cells, or has a large difference in skin condition between both hands, the contact impedance difference may be very large and may make ECG measurement difficult (e.g., impossible in some instances).

Various embodiments disclosed herein may provide a cover-type electronic device and a method for measuring biometric data thereby, wherein various gripping postures for biometric data measurement are supported, and the measurement performance can be maintained no matter what gripping posture the user takes.

Various embodiments disclosed herein may provide a cover-type electronic device and a method for measuring biometric data thereby, wherein an electrode area for biometric data measurement is secured such that design flexibility can be improved.

Various embodiments disclosed herein may provide a cover-type electronic device and a method for measuring biometric data thereby, wherein difficulty in biometric data measurement due to the user's physical condition, or measurement performance degradation can be improved.

A cover-type electronic device according to various embodiments may include a cover-type housing, an electrode set including a plurality of electrodes disposed outside (e.g., on an exterior surface of) the housing, and a printed circuit board electrically connected to the plurality of electrodes. A driving circuit in the printed circuit board may be configured to acquire sensing information, identify a user's gripping posture, which is one of a plurality of gripping postures for measurement of designated biometric data, based on the sensing information, and switch a connection state of the electrode set, based on the gripping posture such that the designated biometric data is measured in the switched state.

A cover-type electronic device according to various embodiments may include a cover-type housing, an electrode set including a plurality of electrodes disposed outside the housing, and a printed circuit board electrically connected to the plurality of electrodes. A driving circuit in the printed circuit board may be configured to identify information corresponding to a contact impedance difference between both hands of the user from a biosignal detected through the electrode set, and switch a connection state of the electrode set, based on the information corresponding to the contact impedance difference between both hands of the user such that the user's biometric data is measured in the switched state.

A biometric data measuring method by a cover-type electronic device including an electrode set according to various embodiments may include acquiring sensing information, identifying one of user's gripping postures for measurement of designated biometric data, based on the sensing information, and switching the electrode set, based on the gripping posture such that the designated biometric data is measured in the switched state.

A biometric data measuring method by a cover-type electronic device including an electrode set according to various embodiments may include identifying information corresponding to a contact impedance difference between both hands of the user by using a biosignal detected through the electrode set, and switching a connection state of the electrode set, based on the information corresponding to the contact impedance difference between both hands of the user such that the user's biometric data is measured in the switched state.

According to various embodiments, various gripping postures for biometric data measurement are supported, and the measurement performance can be maintained no matter what gripping posture the user takes.

According to various embodiments, an electrode area for biometric data measurement may be secured such that design flexibility can be improved.

According to various embodiments, difficulty in biometric data measurement due to the user's physical condition, or measurement performance degradation may be improved.

Various other advantageous effects identified explicitly or implicitly through the disclosure may be provided.

Before undertaking the detailed description below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a configuration of a cover-type electronic device according to an embodiment;

FIG. 2 is an exploded perspective view illustrating a fastening structure between a cover-type electronic device and an electronic device according to an embodiment;

FIG. 3 is a block diagram of a driving circuit in a printed circuit board in a cover-type electronic device according to an embodiment;

FIG. 4A illustrates an electrode connection state in a cover-type electronic device according to a first gripping posture according to an embodiment;

FIG. 4B illustrates an electrode connection state in a cover-type electronic device according to a second gripping posture according to an embodiment;

FIG. 4C illustrates an electrode connection state in a cover-type electronic device according to a third gripping posture according to an embodiment;

FIG. 4D illustrates an electrode connection state in a cover-type electronic device according to a fourth gripping posture according to an embodiment;

FIG. 4E illustrates an electrode connection state in a cover-type electronic device according to a fifth gripping posture according to an embodiment;

FIG. 4F illustrates an electrode connection state in a cover-type electronic device according to a sixth gripping posture according to an embodiment;

FIG. 5A exemplarily illustrates a method for measuring impedance between electrodes for identifying a third gripping posture or a fourth gripping posture in the cover-type electronic device according to an embodiment;

FIG. 5B exemplarily illustrates a method for measuring impedance between electrodes for identifying a fifth gripping posture or a sixth gripping posture in the cover-type electronic device according to an embodiment;

FIG. 6A is an example illustrating an electrode connection state for compensating for a contact impedance difference in a cover-type electronic device according to an exemplary embodiment;

FIG. 6B is an example illustrating an electrode connection state for compensating for a contact impedance difference in a cover-type electronic device according to an embodiment;

FIG. 7 is a flowchart showing a biometric data measuring method by a cover-type electronic device according to an embodiment;

FIG. 8 is a flowchart showing a biometric data measuring method using a cover-type electronic device and an electronic device according to an embodiment:

FIG. 9 is a flowchart showing a biometric data measuring method by a cover-type electronic device according to another embodiment;

FIG. 10 is a flowchart showing a biometric data measuring method using a cover-type electronic device and an electronic device according to another embodiment; and

FIG. 11 is a flowchart showing a biometric data measuring method using a cover-type electronic device and an electronic device according to another embodiment.

FIG. 12 is a block diagram of an electronic device in a network environment, according to various embodiments.

### DETAILED DESCRIPTION

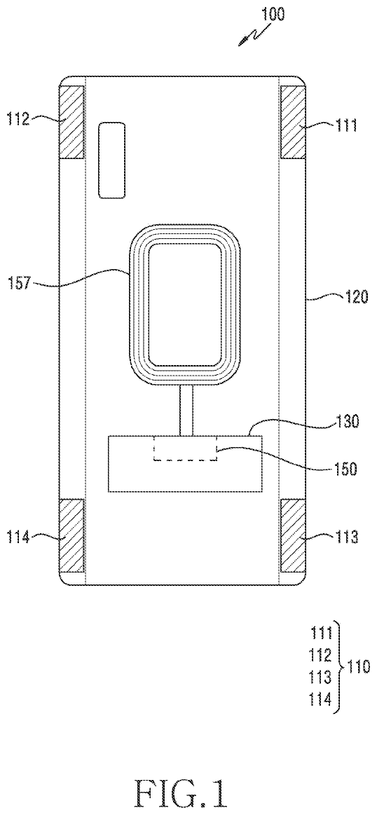

FIGS. 1 through 12, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

Hereinafter, various embodiments are described with reference to the accompanying drawings.

In describing various embodiments of the document, a cover-type electronic device having a structure that can be fastened to a smartphone type electronic device is illustrated and described, but the scope of the embodiments is not limited thereto. For example, a cover-type electronic device according to various embodiments may be of various types of devices configured to be fastened to an electronic device having portability or mobility (e.g., a smartphone, a flexible smartphone, a mobile terminal, a laptop computer, a personal digital assistant (PDA), a netbook, a mobile internet device (MID), an ultra-mobile personal computer (UMPC), a tablet personal computer (a tablet PC), and a navigation).

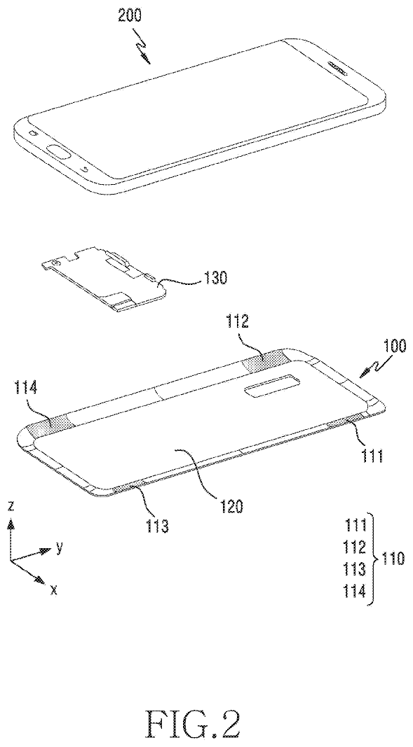

FIG. 1 illustrates a configuration of a cover-type electronic device according to an embodiment. For example, FIG. 2 may correspond to a rear surface of the cover-type electronic device 100. FIG. 2 is an exploded perspective view illustrating a fastening structure between a cover-type electronic device and an electronic device according to an embodiment.

Referring to FIGS. 1 and 2, the cover-type electronic device 100 according to an embodiment may include a housing 120, an electrode set 110, and a printed circuit board 130. A driving circuit 150 may be disposed on the printed circuit board 130.

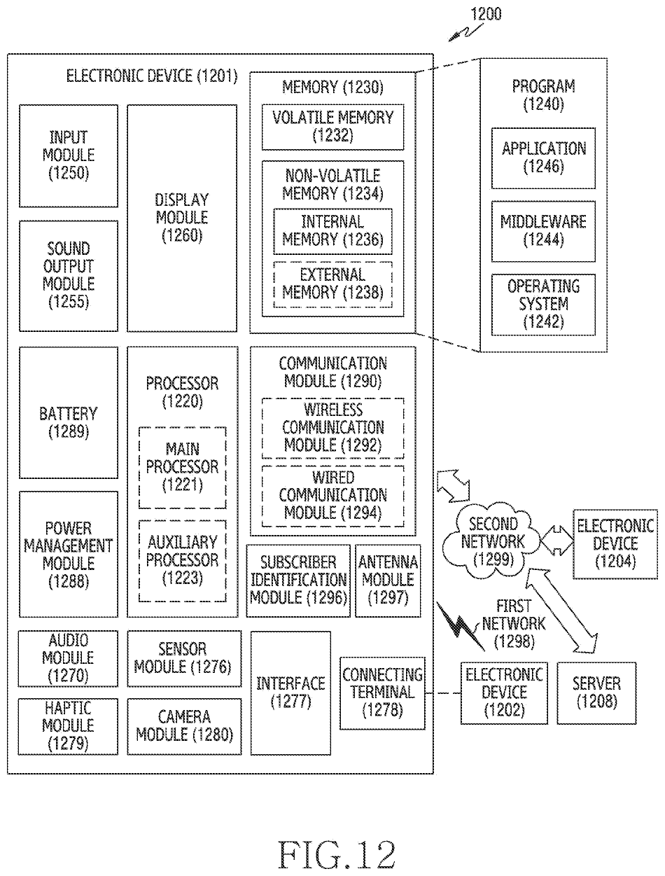

According to an embodiment, the cover-type electronic device 100 may be a device that can be fastened to the electronic device 200 (e.g., a smartphone). For example, the cover-type electronic device 100 as an accessory of the electronic device 200 may have a structure capable of being fastened to the electronic device 200. For example, the electronic device 200 as a fastening target of the cover-type electronic device 100 may have a structure corresponding to an electronic device 1201 illustrated in FIG. 12.

According to an embodiment, the housing 120 of the cover-type electronic device 100 may be a cover type. The housing 120 may have a structure capable of being fastened to the electronic device 200. The housing 120 may be referred to as a cover, frame, or case. For example, the housing 120 may be configured to cover at least a portion of the electronic device 200 (e.g., at least a portion of a front surface, a side surface, and/or a rear surface of the electronic device 200). For example, the housing 120 may be configured to be attachable to or detachable from the electronic device 200. For example, the housing 120 may have a structure into which a portion of the electronic device 200 (e.g., at least a portion of a front surface, a side surface, and/or a rear surface) can be fitted to be mounted. The electronic device 200 may be stably placed in a space formed by the housing 120 to be fastened to the cover-type electronic device 100.

The housing 120 may be configured in various forms. For example, the housing 120 may be in the form of a side plate that surrounds and covers the outer side of the electronic device 200, a rear plate that covers at least a portion of the rear surface of the electronic device 200, a front plate that covers the bezel region of the electronic device 200, and/or a combination of at least some thereof.

FIGS. 1 and 2 show examples of a case in which the housing 120 of the cover-type electronic device 100 covers the side surface and the rear surface of the electronic device 200 (e.g., a smartphone).

For example, as illustrated, the housing 120 may include a side plate that covers the side surface of the electronic device 200 (e.g., a smartphone), and a rear plate that covers most portion of the rear surface of the electronic device 200, except for a partial region (e.g., a camera region in which the rear camera lens of the electronic device 200 is exposed).

The illustrated shape or structure of the housing 120 is only an example, and the scope of the embodiments is not limited thereto.

In various embodiments, the housing 120 of the cover-type electronic device 100 may be implemented in various shapes or structures that can be fastened to an electronic device (e.g., the electronic device 200) and can be gripped by a user. The housing 120 of the cover-type electronic device 100 may be variously changed, modified, applied and/or expanded in response to the shape (or appearance) or structure of the electronic device (e.g., the electronic device 200) to be fastened.

For example, the cover-type electronic device 100 may be manufactured to fit the external shape of the electronic device 200 (e.g., a smartphone) to be fastened and may be used to cover (or protect) the electronic device 200 or to improve an aesthetic sense. The cover-type electronic device 100 may have a different shape or structure depending on the model (or standard) of the electronic device 200 to be fastened.

As another example, in case that the electronic device to be fastened is a slidable smartphone, the cover-type electronic device may include a slidable structure that is variable in response to sliding-in and/or sliding-out operations of the slidable smartphone. For example, the housing of the cover-type electronic device may include a first plate and a second plate slidably fastened to the first plate.

As still another example, in case that the electronic device to be fastened is a foldable smartphone, the cover-type electronic device may include a foldable structure that is variable in response to folding and/or unfolding operations of the foldable smartphone. For example, the housing of the cover-type electronic device may include a first plate on one side, a second plate on the other side, and a hinge structure disposed between the first plate and the second plate to support folding and/or unfolding.

According to an embodiment, the electrode set 110 may include a plurality of electrodes. For example, the electrode set 110 may include a first electrode 111, a second electrode 112, a third electrode 113, and a fourth electrode 114. The plurality of electrodes may be disposed outside the housing 120.

According to an embodiment, the plurality of electrodes may be dispersedly disposed on both sides (e.g., the left and right and/or up and down) of the housing 120 to enable a user to grip the housing by using both hands. For example, as shown, four electrodes (e.g., the first electrode 111, the second electrode 112, the third electrode 113, and the fourth electrode 114) may be arranged on the outer side surface of the housing 120. For example, two electrodes (e.g., the first electrode 111 and the third electrode 113) may be spaced apart on one side (e.g., the right side) of the housing 120, and two electrodes (e.g., the second electrode 112 and the fourth electrode 114) may be spaced apart on the other side (e.g., the left side) of the housing 120. For example, more electrodes (e.g., four) than the number of electrodes (e.g., three) required for measuring designated biometric data (e.g., electrocardiogram data) may be disposed in the cover-type electronic device 100, and a connection state of the electrodes may be switched in response to a gripping posture of the user.

According to an embodiment, a plurality (e.g., four) of electrodes may be disposed to correspond to each corner of the cover-type electronic device 100 (or the housing 120 of the cover-type electronic device 100). Each of the plurality of electrodes may be located at or adjacent to each corner of the cover-type electronic device 100. For example, in case that the electronic device 200 is a smartphone, each of the four electrodes may be disposed to correspond to each corner of the electronic device 200 having a rectangular shape. For example, four electrodes may be configured outside the housing 120 of the cover-type electronic device 100 to correspond to the shape or structure of the electronic device 200.

According to an embodiment, the printed circuit board 130 may be electrically connected to the electrode set 110 (or a plurality of electrodes). For example, the printed circuit board 130 may be a flexible printed circuit board. For example, the printed circuit board 130 may be inserted into the housing 120, attached to the inner surface of the housing 120, or integrally formed with the housing 120.

According to an embodiment, the printed circuit board 130 may include a driving circuit 150. The driving circuit 150 in the printed circuit board 130 may be for measuring biometric data. For example, the driving circuit 150 may be implemented to include one or more integrated circuits (ICs) or to be patterned on a flexible printed circuit board.

According to an embodiment, the cover-type electronic device 100 may perform a function of measuring biometric data while being fastened to the electronic device 200 and/or while being connected to the electronic device 200 via short-range wireless communication (e.g., NFC, Bluetooth, Bluetooth LE, or WiFi direct).

According to an embodiment, the cover-type electronic device 100 and the electronic device 200 may be connected via short-range wireless communication through the driving circuit 150 in the printed circuit board 130. For example, the electronic device 200 may be in a state in which the same is fastened to the housing 120 of the cover-type electronic device 100.

According to an embodiment, the driving circuit 150 in the printed circuit board 130 may perform an electrode switching operation, based on the user's gripping posture (or current gripping posture).

According to an embodiment, the driving circuit 150 may acquire sensing information.

According to an embodiment, the sensing information may include one or more of direction information, impedance information (electrode-electrode impedance) between a plurality of electrodes, and phase information of a user's biosignal (e.g., an electrocardiogram signal).

For example, the sensing information may include direction information. For example, the direction information may be received from the electronic device 200 (e.g., at least one sensor (e.g., an acceleration sensor, a gyro sensor, and/or an image sensor) in the electronic device 200) via short-range wireless communication.

As another example, the sensing information may include impedance information between a plurality of electrodes.

As still another example, the sensing information may include phase information of a user's biosignal (e.g., an electrocardiogram signal).

According to an embodiment, the driving circuit 150 may identify the user's gripping posture (or current gripping posture), which is one of a plurality of gripping postures for measurement of designated biometric data (e.g., electrocardiogram data), based on the sensing information.

According to an embodiment, the plurality of gripping postures may be gripping postures in which a user uses both hands.

According to an embodiment, the driving circuit 150 may switch a connection state of the electrode set 110 (or a plurality of electrodes), based on the user's gripping posture (or the current gripping posture) such that designated biometric data (e.g., electrocardiogram data) is measured in the switched state. Switching the connection state (or the electrode connection state) of the electrode set 110 may be understood as switching the connection state of at least some of the plurality of electrodes in the electrode set 110 or switching the connection state between the electrode set 110 (or the plurality of electrodes) and the driving circuit 150 (e.g., an input terminal of the biometric sensor (biosensor 153)).

According to an embodiment, the driving circuit 150 may measure the designated biometric data for a reference period of time or longer in order to increase measurement accuracy.

According to an embodiment, the driving circuit 150 may transmit the measured designated biometric data to the electronic device 200 via short-range wireless communication. For example, the electronic device 200 may provide a healthcare service using the received designated biometric data. For example, the electronic device 200 may store, observe, or analyze the received designated biometric data (e.g., electrocardiogram data) to diagnose the user's health condition or disease (e.g., myocardial infarction, arrhythmia, or various cardiac diseases such as hyperkalemia), and output the result to a user interface (e.g., a screen or voice) to provide the same to the user.

According to an embodiment, as illustrated, the plurality of electrodes may be respectively disposed to correspond to the corners of the housing 120. In case that the plurality of electrodes are disposed to correspond to the corners of the housing 120, a user's grip and/or contact with the electrodes may be facilitated, and thus usability or functionality may be improved.

According to an embodiment, the cover-type electronic device 100 may enter a measurement mode and perform a biometric data measurement operation in the measurement mode.

For example, the cover-type electronic device 100 may enter the measurement mode in response to sensing a user's contact for a predetermined time period (e.g., 2 to 3 seconds) or longer with respect to the plurality of electrodes.

As another example, the cover-type electronic device 100 may enter a measurement mode in response to an event (e.g., an electrocardiogram measurement event) for starting measurement of designated biometric data, occurring in the electronic device 200. The measurement mode may be terminated in response an event (e.g., an electrocardiogram measurement end event) for terminating measurement of the designated biometric data, occurring in the electronic device 200.

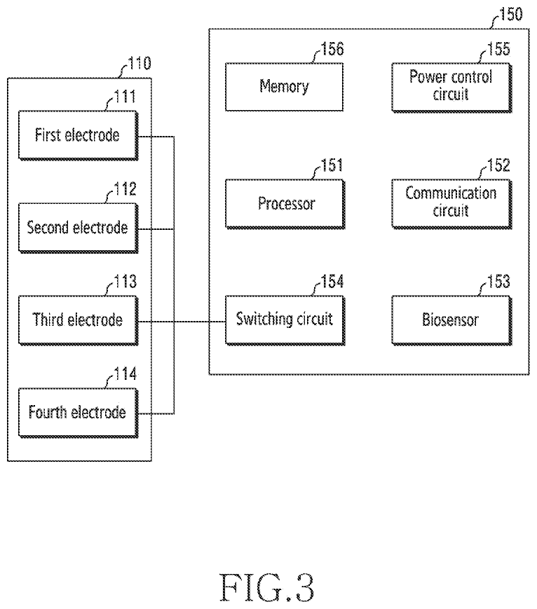

According to an embodiment, the cover-type electronic device 100 (or the housing 120 of the cover-type electronic device 100) may include a coil 157. For example, the coil 157 may be a wireless power receiving coil. The coil 157 may be for receiving wireless power from the electronic device 200. For example, the coil 157 may be electrically connected to a power control circuit 155 of FIG. 3 to be described later or may be included in the power control circuit 155. For example, the coil 157 may have a different shape and may be disposed at different positions according to the type of the electronic device 200 (e.g., a smartphone) to be fastened.

According to an embodiment, the cover-type electronic device 100 may operate in conjunction with the electronic device 200 or utilize a resource of the electronic device 200 during a biometric data measurement operation. For example, the electronic device 200 (e.g., a smartphone) may correspond to the electronic device 1201 of FIG. 12. For example, the cover-type electronic device 100 may receive and use wireless power from the electronic device 200 to perform a biometric data measurement operation or may utilize at least one sensor (e.g., an acceleration sensor, a gyro sensor, and/or image sensor) in the electronic device 200.

In various embodiments of the document, a case where the designated biometric data as a measurement target is electrocardiogram data is mainly exemplified, but the type of the designated biometric data is not limited thereto. For example, the designated biometric data may also be information on any one of a heart rate, an electrodermal activity (EDA), and a bioelectrical impedance. For example, the designated biometric data may be biometric data that can be measured in a gripping state using both hands (or the fingers of both sides) of a user. As another example, the designated biometric data may be biometric data that can be measured using a plurality (e.g., three or four) of electrodes.

In case that the types of biosignals as measurement targets increase, the range of measurement postures to be considered is widened, and accordingly, it may be difficult to specify a measurement posture of a user. On the other hand, in case that the measurement target is one type of designated biometric data, compared to the case where various types of biosignals become measurement targets at the same time, the current gripping posture among a plurality of gripping postures related to the designated biometric data can be easily and accurately specified, and the usability or functionality of the measurement function may be improved.

According to an embodiment, the driving circuit 150 in the printed circuit board 130 may perform an electrode switching operation to compensate for a contact impedance difference between both hands of the user. The contact impedance may refer to an impedance between the user's skin and the electrode.

In an embodiment, the driving circuit 150 may identify information (e.g., a direct current offset of a biosignal) corresponding to a contact impedance difference between both hands of the user from the biosignal sensed through the electrode set 110. The driving circuit 150 may switch the connection state of the electrode set, based on information corresponding to the contact impedance difference between both hands of the user.

In an embodiment, the electrode set 110 may include a positive electrode, a negative electrode, and at least one electrode in a short-circuit state.

For example, one hand of a user may be in contact with an electrode (e.g., one of the positive and negative electrodes) on one side and the other hand of the user may be in contact with an electrode (e.g., the other of the positive and negative electrodes) on the other side. In this state, the driving circuit 150 may analyze the contact impedance difference between both hands of the user by using the biosignal sensed through the positive electrode and the negative electrode and may perform electrode switching in case that the contact impedance difference is greater than a predetermined value.

In an embodiment, the driving circuit 150 may compensate for a contact impedance difference between both hands of the user by electrically connecting at least one electrode in a short-circuit state to a positive electrode or a negative electrode, by an electrode switching operation.

The driving circuit 150 may increase the electrode contact area by connecting two or more electrodes having a greater impedance to each other by switching to compensate for the contact impedance. For example, in case that the impedance of an electrode (e.g., one of the positive electrode and the negative electrode) on one side is greater than the impedance of an electrode (e.g., the other one of the positive electrode and the negative electrode) on the other side, at least one electrode in a short-circuit state may be electrically connected to the electrode on one side. As another example, in case that the impedance of the electrode on the other side is greater than the impedance of the electrode on one side, the at least one electrode may be electrically connected to the electrode on the other side.

In a case in which a contact impedance difference between both hands of a user is great during measurement, biometric data measurement may not be possible due to an increase in a DC component. The contact impedance difference may be inversely proportional to an electrode contact area. In case that the electrode contact area is increased by switching, the contact impedance difference can be reduced.

In an embodiment, to increase measurement accuracy, the driving circuit 150 may detect a biosignal for a first reference time or longer (for contact impedance difference analysis) and measure biometric data for a second reference time or longer (for measurement).

In an embodiment, the switching operation for compensating for the contact impedance difference between both hands of the user may be performed independently of the switching operation based on the user's gripping posture. Alternatively, the two switching operations may be performed in parallel or sequentially.

According to an embodiment, the cover-type electronic device 100 (or the driving circuit 150) may perform the electrode switching operation in consideration of both the user's gripping posture and the contact impedance difference between both hands of the user. For example, the cover-type electronic device 100 may perform an electrode switching operation of changing at least some of the positive electrode, the negative electrode, and the ground electrode in the electrode set 110, based on the user's gripping posture. Additionally, the cover-type electronic device 100 may electrically connect at least one electrode in a short-circuit state in the electrode set 110 to the changed positive electrode or negative electrode to compensate for a contact impedance difference between both hands of the user.

FIG. 3 is a block diagram of a driving circuit in a printed circuit board in a cover-type electronic device according to an embodiment.

In an embodiment, the driving circuit 150 in the printed circuit board 130 may perform an electrode switching operation, based on a user's gripping posture (or current gripping posture).

Referring to FIG. 3, the cover-type electronic device 100 according to an embodiment may include a printed circuit board 130. The driving circuit 150 may be disposed on the printed circuit board 130. The driving circuit 150 in the printed circuit board 130 may include at least one processor 151, a communication circuit 152, a biosensor 153, a switching circuit 154, and a power control circuit 155. The driving circuit 150 may further include a memory 156. In some embodiments, some of the components may be omitted from the driving circuit 150, at least some of the components may be integrated, or other components may be additionally included.

In an embodiment, the at least one processor 151 may be electrically connected to at least some of the communication circuit 152, the biosensor 153, the switching circuit 154, the power control circuit 155, and the memory 156. The at least one processor 151 may operate in conjunction with at least some of the communication circuit 152, the biosensor 153, the switching circuit 154, the power control circuit 155, and the memory 156 or may control at least some thereof.

According to an embodiment, the communication circuit 152 may be for short-range wireless communication with the electronic device 200. The communication circuit 152 may support establishment of a channel for short-range wireless communication (e.g., near field communication (NFC), Bluetooth, Bluetooth low energy (BLE), wireless fidelity (WiFi) direct or infrared data association (IrDA)) or communication via the established communication channel. The communication circuit 152 may perform short-range wireless communication function autonomously or under the control of at least one processor 151.

According to an embodiment, the biometric sensor 153 may be for measuring designated biometric data (e.g., electrocardiogram data). The designated biometric data of a user gripping the cover-type electronic device 100 (or the plurality of electrodes of the cover-type electronic device 100) may be measured through the biosensor 153. The biosensor 153 may measure biometric data by using output from at least some of the plurality of electrodes included in the electrode set 110.

For example, the biosensor 153 may be an electrocardiography (ECG) sensor, a bioelectrical impedance analysis (BIA) sensor, an electrodermal activity (EDA) sensor, or other biometric sensor. The reference number 153 refers to the biosensor 153, generally, and also refers to the ECG sensor 153, in some specific embodiments. For example, the designated biometric data measurable through the biosensor 153 may be one of electrocardiogram, bioimpedance, heart rate, blood pressure, blood oxygen saturation, stress, and/or a galvanic skin response.

According to an embodiment, the biosensor 153 may be connected to the electrode set 110 (or a plurality of electrodes constituting the electrode set 110) through the switching circuit 154. For example, the biosensor 153 may differentiate, amplify, and/or filter an electrical signal coming from a plurality of electrodes through the switching circuit 154, and measure the biometric data by converting the filtered signal into digital data. The biometric data measured by the biosensor 153 may be transmitted to the electronic device 200 through the communication circuit 152.

According to an embodiment, the switching circuit 154 may be disposed between the plurality of electrodes and the biosensor 153. The switching circuit 154 may change a connection state (or an electrode connection state) between the plurality of electrodes and the biosensor 153 through a switching operation. The connection state may be understood as an electrical connection state and/or a physical connection state. For example, the switching circuit 154 may include an electrode switching element (e.g., a switching box) having terminals on one side (e.g., input terminals) connected to the plurality of electrodes and terminals on the other side (e.g., output terminals) connected to an input terminal of the biosensor 153. For example, a connection state of each electrode connected to each terminal may be changed (or switched) or each electrode may be turned on or off by an on or off operation for each terminal of the electrode switching element.

According to an embodiment, the power control circuit 155 may receive power supplied from the electronic device 200 fastened to the housing 120 and may drive the cover-type electronic device 100 (e.g., the driving circuit 150 of the cover-type electronic device 100). For example, the power control circuit 155 may receive and use (e.g., charge) wireless power from the electronic device 200.

For example, the power control circuit 155 may receive and use wireless power supplied by BLE or wireless power transmission (WPT) scheme. For example, the power control circuit 155 may be connected to the coil 157 (e.g., a wireless power receiving coil) shown in FIG. 1 and may receive and use power supplied to the coil 157 of the cover-type electronic device 100 from a coil (e.g., a wireless power transmitting coil) of the electronic device 200. In some embodiments, the coil 157 may be integrally formed within the power control circuit 155.

According to an embodiment, the at least one processor 151 may perform signal processing, data transmission, and/or control on the measured biometric data (e.g., electrocardiogram data).

According to an embodiment, the at least one processor 151 may acquire sensing information through the communication circuit 152 or the biosensor 153.

For example, the sensing information may include direction information. For example, the processor 151 may acquire (or receive) the direction information from the electronic device 200. For example, the processor 151 may receive direction information of the electronic device 200 through the communication circuit 152 from the electronic device 200 fastened to the cover-type electronic device 100 or connected to the cover-type electronic device via short-range wireless communication. The direction information of the electronic device 200 may include information on a tilt angle. For example, the electronic device 200 may provide information on the tilt angle of the electronic device 200 sensed through at least one sensor (e.g., an acceleration sensor or a gyro sensor) in the electronic device 200 to the cover-type electronic device 100.

As another example, the sensing information may include at least one of impedance information between the plurality of electrodes and phase information of a user's biosignal. For example, the processor 151 may acquire (or measure) at least one of impedance information between the plurality of electrodes and phase information of a user's biosignal through the biometric sensor 153.

According to an embodiment, the processor 151 may identify the user's gripping posture (or the current gripping posture), which is one of a plurality of gripping postures for measurement of designated biometric data (e.g., electrocardiogram data), based on the sensing information.

For example, reference sensing information for a plurality of gripping postures may be stored in advance in order to identify the gripping posture. Current sensing information may be acquired according to the gripping of a plurality of electrodes. One gripping posture among the plurality of gripping postures may be identified based on a comparison between the reference sensing information and the current sensing information.

For example, the reference sensing information may include one or more of reference direction information, reference impedance information, and reference phase information. For example, the current sensing information may include one or more of current direction information of the electronic device 200, current impedance information between a plurality of electrodes, and current phase information of a user's biosignal.

According to an embodiment, the at least one processor 151 may switch the connection state of the electrode set 110 (or a plurality of electrodes), based on the identified gripping posture such that designated biometric data is measured in the switched state.

According to an embodiment, the driving circuit 150 in the printed circuit board 130 may perform an electrode switching operation for compensating for contact impedance between both hands of the user.

In an embodiment, the electrode set 110 may include a plurality of electrodes. The plurality of electrodes may include a positive electrode (e.g., one of the first electrode 111, the second electrode 112, the third electrode 113, and the fourth electrode 114), a negative electrode (e.g., another one of the first electrode 111, the second electrode 112, the third electrode 113, and the fourth electrode 114), and at least one electrode (e.g., the other one of the first electrode 111, the second electrode 112, the third electrode 113, and the fourth electrode 114) in a short-circuit state.

The positive electrode may be an electrode electrically connected to a positive terminal of the biosensor 153. The negative electrode may be an electrode is electrically connected to a negative terminal of the biosensor 153. At least one electrode in a short-circuit state may be an electrode short-circuited (or blocked) with the biosensor 153.

In an embodiment, the driving circuit 150 may analyze a contact impedance difference between both hands of the user from the biosignal sensed through the positive electrode and the negative electrode among the plurality of electrodes in the electrode set 110, and switch a connection state (or an electrode connection state) of the electrode set 110 in order to compensate for the contact impedance difference.

In an embodiment, the driving circuit 150 may perform a switching operation in case that the contact impedance difference exceeds a predetermined value (e.g., a designated value among 10 to 100 MΩ).

During switching, at least one electrode in a short-circuit state among the plurality of electrodes may be electrically connected to the positive electrode or the negative electrode. For example, in case that the impedance of an electrode (e.g., one of the positive electrode and the negative electrode) on one side in the electrode set 110 is greater than the impedance of an electrode on the other side, the driving circuit 150 may control the switching circuit 154 such that at least one electrode in a short-circuit state is connected to an electrode on one side.

Accordingly, a plurality (e.g., two) of electrodes may be used as the same electrode (positive electrode or negative electrode) to compensate for the contact impedance difference and may alleviate measurement difficulties or deterioration in measurement performance due to the user's physical condition.

In an embodiment, the driving circuit 150 may measure the user's designated biometric data (e.g., electrocardiogram data) in the switched electrode connection state.

In an embodiment, in order to increase measurement accuracy, the driving circuit 150 may sense a biosignal for a first reference time or longer, analyze a contact impedance difference by using the biosignal, and measure biometric data for a second reference time or longer.

In an embodiment, the driving circuit 150 in the printed circuit board 130 may perform the electrode switching operation in consideration of a user's gripping posture and a contact impedance difference between both hands of the user.

According to an embodiment, the at least one processor 151 may transmit the measured biometric data to the electronic device 200 through the communication circuit 152.

According to an embodiment, the cover-type electronic device 100 may further include a memory 156. The memory 156 may be omitted according to embodiments. For example, the memory 156 may be unnecessary since the measured biometric data is transmitted to the electronic device 200 in real time in a basic operation. As another example, the measured biometric data may be stored in the memory 156 to enable measurement of the biometric data even in a sleep mode of the electronic device 200. The processor 151 may provide the biometric data stored in the memory 156 to the electronic device 200 through the communication circuit 152 according to the switching of the electronic device 200 from the sleep mode to the normal mode.

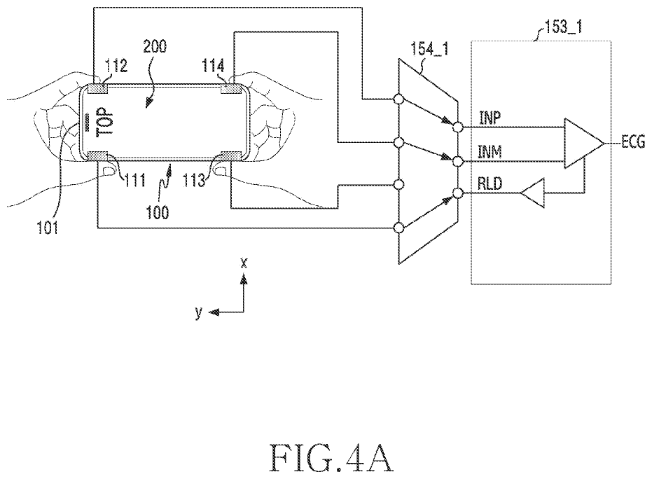

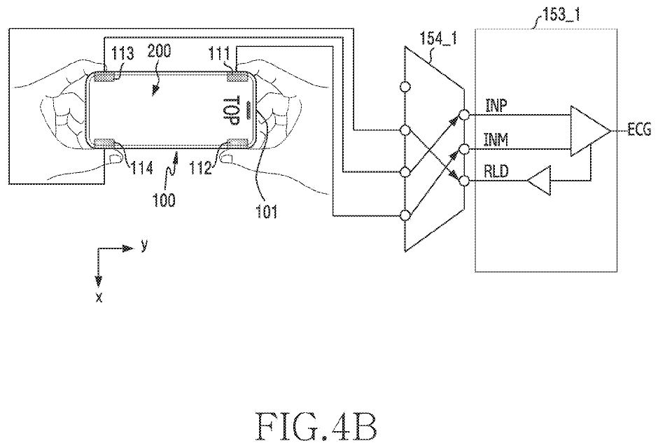

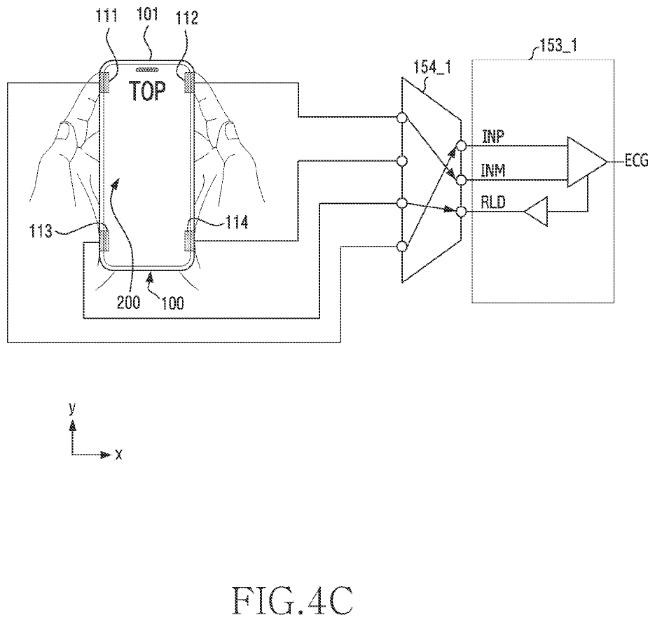

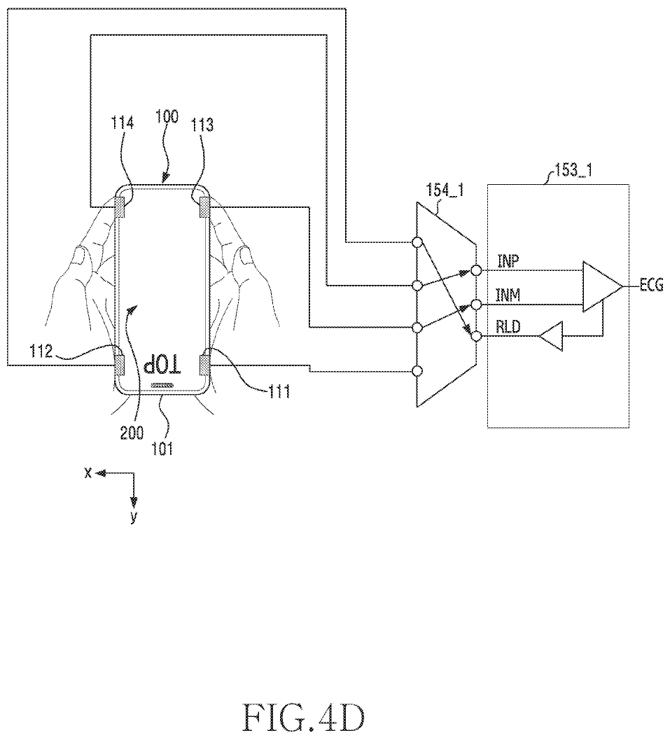

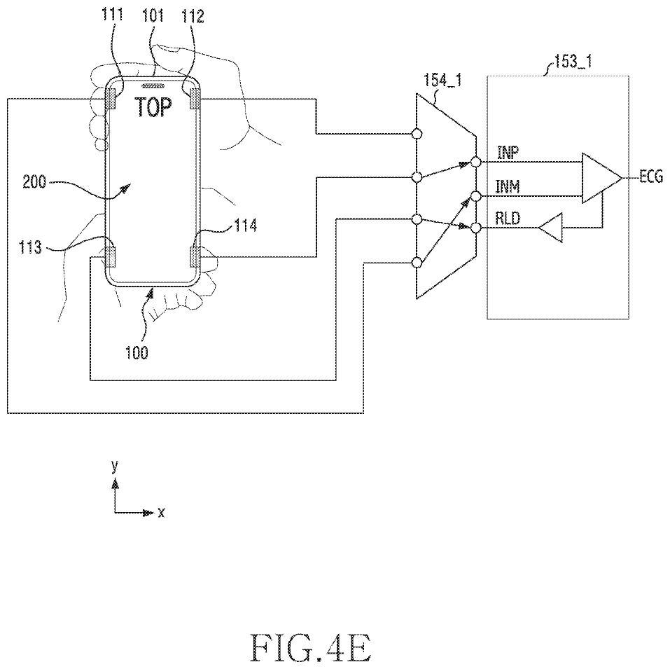

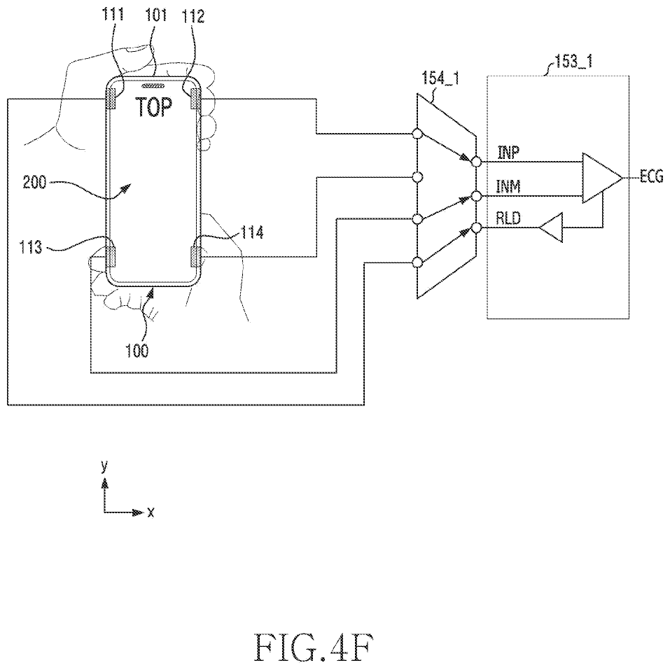

FIGS. 4A to 4F illustrate electrode connection states in a cover-type electronic device according to various gripping postures according to an embodiment. In the examples of FIGS. 4A to 4F, the electrode connection structure of the cover-type electronic device 100 may be for measuring electrocardiogram data. In the examples of FIGS. 4A to 4F, the electronic device 200 may in a state in which the electronic device 200 is fastened to the cover-type electronic device 100 and/or connected to the cover-type electronic device 100 via short-range wireless communication.

As shown in FIGS. 4A to 4F, various gripping postures for measuring designated biometric data may exist, and a comfortable posture may be different according to a user's situation or taste. In case that the electrode connection state is fixed without switching, biometric data may not be measured due to the user's gripping posture different from the basic gripping posture (e.g., the basic gripping posture of FIG. 4A).

According to various embodiments, the cover-type electronic device 100 may switch the electrode connection state, based on a user's gripping posture in order to improve usability or functionality or to allow designated biometric data (e.g., electrocardiogram data) as a measurement target to be normally measured.

According to an embodiment, the cover-type electronic device 100 may identify, using sensing information, a gripping posture of a user gripping the electronic device 200 which is fastened thereto.

For example, the sensing information may include at least one of direction information (e.g., information on a tilt angle) of the electronic device 200 received from the electronic device 200 (e.g., an acceleration sensor of the electronic device 200), impedance information between a plurality of electrodes, and phase information of a user's biosignal (e.g., an electrocardiogram signal).

According to an embodiment, the cover-type electronic device 100 may identify a user's gripping posture (or current gripping posture) as one of a plurality of gripping postures as shown in FIGS. 4A to 4F, based on the sensing information.

According to an embodiment, the cover-type electronic device 100 may switch an electrode connection state to enable normal measurement of designated biometric data (e.g., electrocardiogram data) as a measurement target, based on the identified gripping posture. By switching the electrode connection state to be suitable for the user's gripping posture, the designated biometric data can be measured no matter which gripping posture is taken among the various gripping postures (e.g., the six gripping postures of FIGS. 4A to 4F) adoptable by a user.

According to an embodiment, for switching, the switching circuit 154 may be disposed between the electrode set 110 (or a plurality of electrodes) and the biosensor 153.

FIG. 4A illustrating an electrode connection state in a cover-type electronic device according to a first gripping posture according to an embodiment. The gripping posture illustrated in FIG. 4A may be the first gripping posture. For example, in the first gripping posture, the tilt angle of the electronic device 200 may correspond to +90° (or 90°±offset angle). For example, the first gripping posture may be a basic gripping posture. The illustrated electrode connection state may be a basic electrode connection state corresponding to the basic gripping posture. Reference numeral 101 refers to a top side of the cover-type electronic device 100.

As illustrated, the first gripping posture may be a posture in which a user grips the left and right sides of the cover-type electronic device 100 by using both hands (or the fingers of both hands) while the top side 101 of the cover-type electronic device 100 faces to the left. For example, the first electrode 111 may be in contact with the user's left thumb. The second electrode 112 may be in contact with the user's left index finger. The third electrode 113 may be in contact with the user's right thumb. The fourth electrode 114 may be in contact with the user's right index finger. In the first gripping posture, the third electrode 113 may be short-circuited (or blocked) or not used.

According to an embodiment, the cover-type electronic device 100 may receive sensing information for identification of a gripping posture from the electronic device 200 fastened to the cover-type electronic device 100. For example, the cover-type electronic device 100 may receive information on a tilt angle (e.g., 90°) of the electronic device 200 measured by at least one sensor (e.g., an acceleration sensor) in the electronic device 200 and may identify the user's gripping posture as the first gripping posture, based on the information on the tilt angle.

According to an embodiment, the cover-type electronic device 100 may include an ECG sensor 153_1 configured to measure electrocardiogram data. The ECG sensor 153_1 may be the biosensor 153 shown in FIG. 3 or an example of a component in the biosensor 153. The illustrated ECG sensor 153_1 shows a schematic internal configuration for convenience and may further include one or more other components (e.g., a resistor, an amplifier, a filter) for electrocardiogram data measurement.

According to an embodiment, the cover-type electronic device 100 may include a switching element 154_1 configured to switch an electrode connection state. For example, switching an electrode connection state may be understood as changing or switching a connection state between the plurality of electrodes and the ECG sensor 153_1. The switching element 154_1 may be the switching circuit 154 shown in FIG. 3 or an example of a component in the switching circuit 154. For the switching operation, the switching element 154_1 may be disposed between a plurality of electrodes (the first electrode 111, the second electrode 112, the third electrode 113, and the fourth electrode 114) and the ECG sensor 153_1 (e.g., an input terminal of the ECG sensor 153_1).

The cover-type electronic device 100 (or the driving circuit 150 or the processor 151 of the cover-type electronic device 100) may control the switching element 154_1 based on a user's gripping posture to implement an appropriate electrode connection state in response to the gripping posture. For example, the cover-type electronic device 100 may determine whether the current connection state of the plurality of electrodes corresponds to the current gripping posture. The cover-type electronic device 100 may perform switching in case that the current connection state does not correspond to the current gripping posture. In case that the current connection state corresponds to the current gripping posture, the current connection state may be maintained without switching.

A total of three electrodes (a positive electrode, a negative electrode, and a ground electrode) may be basically required for electrocardiogram measurement. The positive electrode may be connected to the positive terminal (INP) of the ECG sensor 153_1. The negative electrode may be connected to the negative terminal (INM) of the ECG sensor 153_1. The ground electrode may be connected to the ground terminal RLD of the ECG sensor 153_1.

In the first gripping posture, the second electrode 112 may be used as a positive electrode, and the fourth electrode 114 may be used as a negative electrode. The first electrode 111 may be used as a ground electrode to configure a reference voltage of the user's body and to reduce noise. In a state in which the left hand is in contact with the second electrode 112 as a positive electrode and the right hand is in contact with the fourth electrode 114 as a negative electrode, electrocardiogram data according to the voltage difference between both arms (e.g., electrocardiogram lead I as output between the electrodes in contact with both arms).

The ECG sensor 153_1 may be connected to a plurality of electrodes through the switching element 154_1. The ECG sensor 153_1 may differentiate, amplify, and/or filter the user's electrocardiogram signal coming from the plurality of electrodes through the switching element 154_1 to output electrocardiogram data corresponding to the filtered electrocardiogram signal.

In case that the user's gripping posture (or current gripping posture) is the first gripping posture (or a basic gripping posture), as shown, the second electrode 112 may be connected to a positive terminal (INP) of the ECG sensor 153_1, the fourth electrode 114 may be connected to a negative terminal (INM) of the ECG sensor 153_1, and the first electrode 111 may be connected to the ground terminal (RLD) of the ECG sensor 153_1. For example, such an electrode connection state may be a state before the switching operation. As another example, the cover-type electronic device 100 may control the switching element 1541 to implement such an electrode connection state.

FIG. 4B illustrates an electrode connection state in a cover-type electronic device according to the second gripping posture according to an embodiment. The gripping posture illustrated in FIG. 4B may be the second gripping posture. For example, in the second gripping posture, a tilt angle of the electronic device 200 may correspond to −90° (or −90°±offset angle).

As illustrated, the second gripping posture may be a posture in which a user grips the left and right sides of the type electronic device 100 by using both hands (or fingers of both hands) while the top side 101 of the cover-type electronic device 100 faces to the right (opposite side to the first gripping posture). For example, the first electrode 111 may be in contact with the user's right index finger. The second electrode 112 may be in contact with the user's right thumb. The third electrode 113 may be in contact with the user's left index finger. The fourth electrode 114 may be in contact with the user's left thumb.

The cover-type electronic device 100 may identify the user's gripping posture as the second gripping posture, based on information on a tilt angle (e.g., −90°) of the electronic device 200.

In case that the user's gripping posture is the second gripping posture, an electrode connection state may be switched through the switching element 154_1 as shown in FIG. 4B. As shown, the third electrode 113 may be connected to the positive terminal (INP) of the ECG sensor 153_1, the first electrode 111 may be connected to the negative terminal (INM) of the ECG sensor 153_1, and the fourth electrode 114 may be connected to the ground terminal (RLD) of the ECG sensor 153_1. In the second gripping posture, the second electrode 112 may be short-circuited (or blocked) or not used.

FIG. 4C illustrates an electrode connection state in a cover-type electronic device according to a third gripping posture according to an embodiment. The gripping posture illustrated in FIG. 4C may be a third gripping posture. For example, in the third gripping posture, the electronic device 200 may be in a non-tilting state. The tilt angle of the electronic device 200 may correspond to 0° (or 0°±offset angle).

As illustrated, the third gripping posture may be a posture in which a user grips the left and right sides of the cover-type electronic device 100 by using both hands (or fingers or palms of both hands) while the top side 101 of the cover-type electronic device 100 faces upward. For example, the first electrode 111 may be in contact with the user's left index finger. The third electrode 113 may be in contact with the user's left palm. The second electrode 112 may be in contact with the user's right index finger. The fourth electrode 114 may be in contact with the user's right palm.

The cover-type electronic device 100 may identify the user's gripping posture as the third gripping posture, based on information on a tilt angle (e.g., 0°) of the electronic device 200.

In case that the user's gripping posture is the third gripping posture, the electrode connection state may be switched through the switching element 154_1 as shown in FIG. 4C. As shown, the first electrode 111 may be connected to the positive terminal (INP) of the ECG sensor 153_1, the second electrode 112 may be connected to the negative terminal (NM) of the ECG sensor 153_1, and the third electrodes 113 may be connected to the ground terminal (RLD) of the ECG sensor 153_1. In the third gripping posture, the fourth electrode 114 may be short-circuited (or blocked) or not used.

FIG. 4D illustrates an electrode connection state in a cover-type electronic device according to a fourth gripping posture according to an embodiment. The gripping posture illustrated in FIG. 4D may be a fourth gripping posture. For example, in the fourth gripping posture, the tilt angle of the electronic device 200 may correspond to 180° (or 180°±offset angle).

As shown, the fourth gripping posture may be a posture in which a user grips the left and right sides of the cover-type electronic device 100 by using both hands (or fingers or palms of both hands) while the top side 101 of the cover-type electronic device 100 faces downward. For example, the first electrode 111 may be in contact with the user's right palm. The third electrode 113 may be in contact with the user's right index finger. The second electrode 112 may be in contact with the user's left palm. The fourth electrode 114 may be in contact with the user's left index finger.

The cover-type electronic device 100 may identify the user's gripping posture as the fourth gripping posture, based on information on a tilt angle (e.g., 180°) of the electronic device 200.

In case that the user's gripping posture is the fourth gripping posture, the electrode connection state may be switched through the switching element 154_1 as shown in FIG. 4D. As shown, the fourth electrode 114 may be connected to the positive terminal (INP) of the ECG sensor 153_1, the third electrode 113 may be connected to the negative terminal (INM) of the ECG sensor 153_1, and the second electrode 112 may be connected to the ground terminal RLD of the ECG sensor 153_1. In the fourth gripping posture, the first electrode 111 may be short-circuited (or blocked) or not used.

FIG. 4E illustrates an electrode connection state in a cover-type electronic device according to a fifth gripping posture according to an embodiment. The gripping posture illustrated in FIG. 4E may be the fifth gripping posture. For example, in the fifth gripping posture, the electronic device 200 may be in a non-tilting state. The tilt angle of the electronic device 200 may correspond to 0° (or 0°±offset angle).

As illustrated, the fifth gripping posture may be a posture in which a user grips the upper and lower sides of the cover-type electronic device 100 by using both hands while the top side 101 of the cover-type electronic device 100 faces upward. The fifth gripping posture may be a posture in which the user's right hand grips the top side of the cover-type electronic device 100 and the user's left hand grips the bottom side of the cover-type electronic device 100.

For example, the second electrode 112 may be in contact with the user's right palm. The first electrode 111 may be in contact with the user's right finger. The third electrode 113 may be in contact with the user's left palm. The fourth electrode 114 may be in contact with the user's left finger.

In an embodiment, the cover-type electronic device 100 may identify the user's gripping posture as the fifth gripping posture, based on the information on a tilt angle (e.g., 0°) of the electronic device 200 and the impedance information between the plurality of electrodes.

The tilt angle (e.g., 0°) according to the fifth gripping posture may be the same as the tilt angle (e.g., 0°) according to the third gripping posture.

The second impedance between the plurality of electrodes according to the fifth gripping posture (e.g., the impedance between the first electrode 111 and the second electrode 112 or the impedance between the third electrode 113 and the fourth electrode 114) may appear different from the first impedance between the plurality of electrodes according to the third gripping posture (e.g., the impedance between the first electrode 111 and the second electrode 112 or the impedance between the third electrode 113 and the fourth electrode 114).

For example, in case that the tilt angle of the electronic device 200 is 0°, the cover-type electronic device 100 may distinguish the third gripping posture and the fifth gripping posture by comparing the first impedance and the second impedance.

In case that the user's gripping posture is the fifth gripping posture, the electrode connection state may be switched through the switching element 154_1 as shown in FIG. 4E. As shown, the fourth electrode 114 may be connected to the positive terminal (INP) of the ECG sensor 153_1, the first electrode 111 may be connected to the negative terminal (INM) of the ECG sensor 153_1, and the three electrodes 113 may be connected to the ground terminal (RLD) of the ECG sensor 153_1. In the fifth gripping posture, the second electrode 112 may be short-circuited (or blocked) or not used.

FIG. 4F illustrates an electrode connection state in a cover-type electronic device according to a sixth gripping posture according to an embodiment. The gripping posture illustrated in FIG. 4F may be the sixth gripping posture. For example, in the sixth gripping posture, the electronic device 200 may be in a non-tilting state. The tilt angle of the electronic device 200 may correspond to 0° (or 0°±offset angle).

As illustrated, the sixth gripping posture may be a posture in which a user grips the upper and lower sides of the cover-type electronic device 100 by using both hands while the top side 101 of the cover-type electronic device 100 faces upward. The sixth gripping posture may be a posture in which the user's left hand grips the top side of the cover-type electronic device 100 and the user's right hand grips the bottom side of the cover-type electronic device 100.

For example, the second electrode 112 may be in contact with the user's left finger. The first electrode 111 may be in contact with the user's left palm. The third electrode 113 may be in contact with the user's right finger. The fourth electrode 114 may be in contact with the user's right palm.

In an embodiment, the cover-type electronic device 100 may identify the user's gripping posture as the sixth gripping posture, based on the information on the tilt angle (e.g., 0°) of the electronic device 200 and the phase information of the biosignal.

The tilt angle (e.g., 0°) according to the sixth gripping posture may be the same as the tilt angle (e.g., 0°) according to the third gripping posture or the fifth gripping posture.

A first phase (e.g., a normal state) of the biosignal (e.g., output waveform of the ECG sensor 153_1) according to the sixth gripping posture may appear different from a second phase (e.g., a reversed state) of the biosignal (e.g., output waveform of the ECG sensor 153_1) according to the third gripping posture or the fifth gripping posture.

For example, in case that the tile angle of the electronic device 200 is 0°, the cover-type electronic device 100 may identify the user's gripping posture as the sixth gripping posture by comparing the first phase and the second phase.

In case that the user's gripping posture is the sixth gripping posture, the electrode connection state may be switched through the switching element 154_1 as shown in FIG. 4F. As shown, the second electrode 112 may be connected to the positive terminal (NP) of the ECG sensor 153_1, the third electrode 113 may be connected to the negative terminal (NM) of the ECG sensor 153_1, and the first electrode 11 may be connected to the ground terminal (RLD) of the ECG sensor 153_1. In the sixth gripping posture, the fourth electrode 114 may be short-circuited (or blocked) or not used.

As an example, the cover-type electronic device 100 may identify, though at least one sensor (e.g., an acceleration sensor or an image sensor) in the electronic device 200, the first gripping posture of FIG. 4A, the second gripping posture of FIG. 4B, the third gripping posture of and FIG. 4C, and the fourth gripping posture of FIG. 4D. For example, the cover-type electronic device 100 may identify a user's gripping posture as any one among the first gripping posture to the fourth gripping posture, based on the tilt angle (e.g., 0° for the third gripping posture and 180° for the fourth gripping posture) detected through at least one sensor in the electronic device 200. For example, in case that the tilt angle is 90°, the user's gripping posture may be the first gripping posture. In case that the tilt angle is −90°, the user's gripping posture may be the second gripping posture. In case that the tilt angle is 0°, the user's gripping posture may be the third gripping posture. In case that the tilt angle is 180°, the user's gripping posture may be the fourth gripping posture.

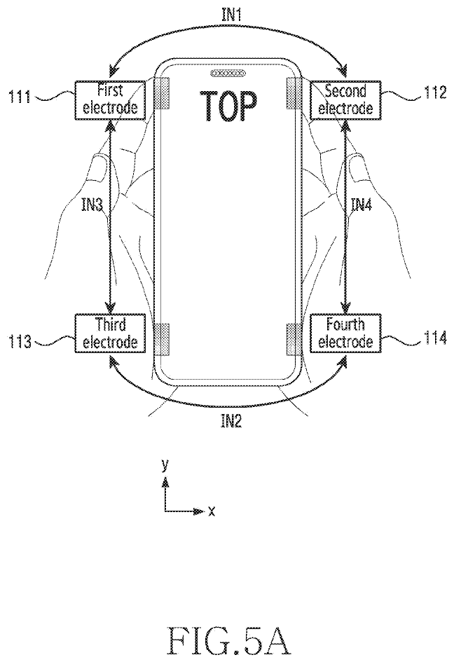

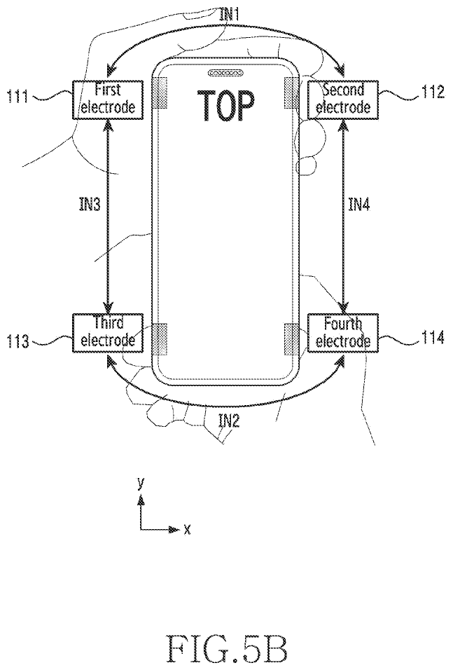

FIG. 5A exemplarily illustrates a method for measuring impedance between electrodes for identifying a third gripping posture or a fourth gripping posture in the cover-type electronic device according to an embodiment.

In an embodiment, the cover-type electronic device 100 may identify the user's gripping posture based on impedance information between the plurality of electrodes.

For example, in the illustrated third gripping posture, the impedance (IN1) between the first electrode 111 and the second electrode 112 or the impedance (IN2) between the third electrode 113 and the fourth electrode 114 may be greater than the impedance (IN3) between the first electrode 111 and the third electrode 113 or the impedance (IN4) between the second electrode 112 and the fourth electrode 114.

Likewise, in the case of the fourth gripping posture, the impedance (IN1) between the first electrode 111 and the second electrode 112 or the impedance (IN2) between the third electrode 113 and the fourth electrode 114 may be greater than the impedance (IN3) between the first electrode 111 and the third electrode 113 or the impedance (IN4) between the second electrode 112 and the fourth electrode 114.

For example, the cover-type electronic device 100 may identify a gripping posture by comparing impedances between a plurality of electrodes. For example, the impedance (IN1) between the first electrode 111 and the second electrode 112 exceeds the impedance (IN3) between the first electrode 111 and the third electrode 113 or the impedance (IN4) between the second electrode 112 and the fourth electrode 114, or the impedance (IN2) between the third electrode 113 and the fourth electrode 114 exceeds the impedance (IN3) between the first electrode 111 and the third electrode 113 or the impedance (IN4) between the second electrode 112 and the fourth electrode 114, the user's gripping posture may be either of the third gripping posture or the fourth gripping posture. The cover-type electronic device 100 may identify a user's gripping posture as one of the third gripping posture and the fourth gripping postures by using information on impedance between a plurality of electrodes and additional information (e.g., information on a tilt angle and/or phase information of a biosignal).

FIG. 5B exemplarily illustrates a method for measuring impedance between electrodes for identifying a fifth gripping posture or a sixth gripping posture in the cover-type electronic device according to an embodiment.

For example, in the illustrated sixth gripping posture, the impedance (IN1) between the first electrode 111 and the second electrode 112 or the impedance (IN2) between the third electrode 113 and the fourth electrode 114 may be smaller than the impedance (IN3) between the first electrode 111 and the third electrode 113 or the impedance (IN4) between the second electrode 112 and the fourth electrode 114.

Likewise, in the case of the fifth gripping posture, the impedance (IN1) between the first electrode 111 and the second electrode 112 or the impedance (IN2) between the third electrode 113 and the fourth electrode 114 may be smaller than the impedance (IN3) between the first electrode 111 and the third electrode 113 or the impedance (IN4) between the second electrode 112 and the fourth electrode 114.

For example, the cover-type electronic device 100 may identify a gripping posture by comparing impedances between a plurality of electrodes. For example, the impedance (IN1) between the first electrode 111 and the second electrode 112 is smaller than the impedance (IN3) between the first electrode 111 and the third electrode 113 or the impedance (IN4) between the second electrode 112 and the fourth electrodes 114, or the impedance (IN2) between the third electrode 113 and the fourth electrode 114 is smaller than the impedance (IN3) between the first electrode 111 and the third electrode 113 or the impedance (IN4) between the second electrode 112 and the fourth electrode 114, the user's gripping posture may be either of the fifth gripping posture and the sixth gripping posture. The cover-type electronic device 100 may identify a user's gripping posture as any one of the fifth gripping posture and the sixth gripping posture by using the information on the impedance between a plurality of electrodes and additional information (e.g., information on a tilt angle and/or phase information of a biosignal).

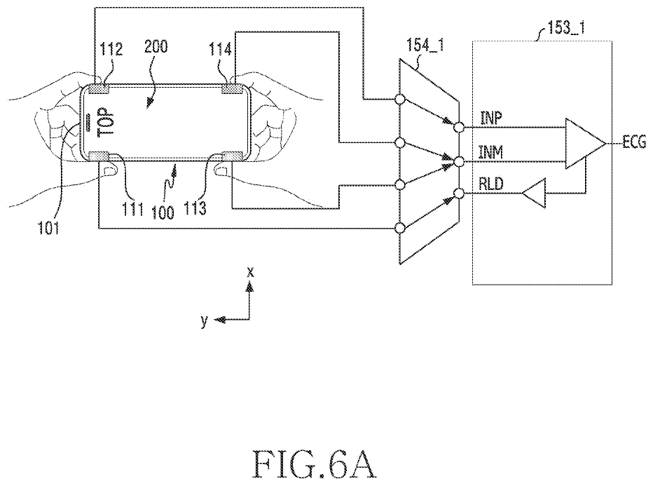

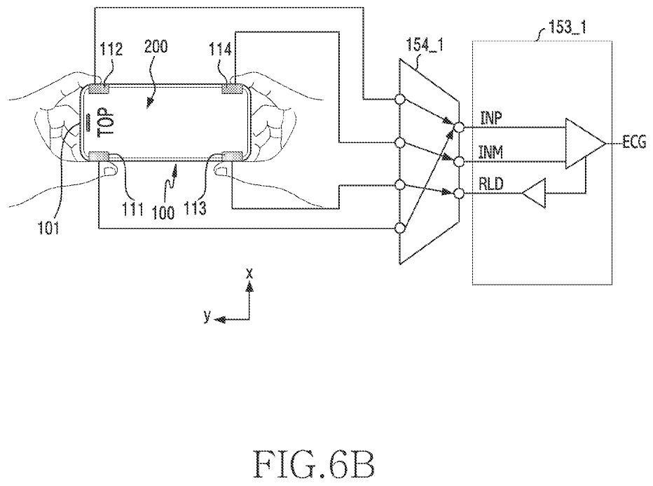

FIGS. 6A and 6B are examples illustrating an electrode connection state for compensating for a contact impedance difference in a cover-type electronic device according to an exemplary embodiment.

In an embodiment, the cover-type electronic device 100 may include an ECG sensor 153_1 configured to measure electrocardiogram data, and a switching element 154_1 configured to switch an electrode connection state. The ECG sensor 153_1 may be the biosensor 153 shown in FIG. 3 or an example of a component in the biosensor 153. The switching element 154_1 may be the switching circuit 154 shown in FIG. 3 or an example of a component in the switching circuit 154. For the switching operation, the switching element 154_1 may be disposed between a plurality of electrodes (e.g., the first electrode 111, the second electrode 112, the third electrode 113, and the fourth electrode 114) and the ECG sensor 153_1 (e.g., an input terminal of the ECG sensor 153_1).

The cover-type electronic device 100 (or the driving circuit 150 or the processor 151 of the cover-type electronic device) may implement an appropriate electrode connection state for compensating for a contact impedance difference by controlling the switching element 154_1.

For example, as shown in FIG. 4A, in the basic electrode connection state, the terminals (INP, INM, and RLD) of the ECG sensor 153_1 may be connected one by one (or one-to-one) to electrodes (e.g., the first electrode 111, the second electrode 112, and the fourth electrode 114) disposed in the electrode set 110 of the cover-type electronic device 100. The second electrode 112 may be connected to the positive terminal (INP) and used as a positive electrode. The fourth electrode 114 may be connected to the negative terminal (INM) and used as a negative electrode. The first electrode 111 may be connected to the ground terminal (RLD) and used as a ground electrode. The electrode set 110 may include at least one electrode (e.g., the third electrode 113) in a short-circuit state.

For example, as shown in FIG. 4A, the user's left hand may be in contact with the second electrode 112 serving as a positive electrode, and the user's right hand may be in contact with the fourth electrode 114 serving as a negative electrode.

For example, to compensate for a contact impedance difference between both hands of the user, the basic electrode connection state as shown in FIG. 4A may be switched to the electrode connection state as shown in FIG. 6A or the electrode connection state as shown in FIG. 6B. Through switching, the plurality (e.g., two) of electrodes may be used as the same electrode (e.g., a positive electrode or a negative electrode) to compensate for the contact impedance difference between both hands of the user.

The cover-type electronic device 100 may analyze the contact impedance difference between both hands of the user by using the electrocardiogram signal detected through the positive electrode and the negative electrode of the electrode set 110.

As an example, in case that, as a result of analyzing the contact impedance difference from the electrocardiogram signal detected in the basic electrode connection state as shown in FIG. 4A, the contact impedance difference is greater than a configured value and the impedance of the negative electrode (e.g., the fourth electrode 114 of FIG. 4A) is greater than the impedance of the positive electrode (e.g., the second electrode 112 of FIG. 4A), the driving circuit 150 (or the processor 151) of the cover-type electronic device 100 may control the switching element 154_1 to change the electrode connection state as shown in FIG. 6A so as to compensate for the contact impedance. Accordingly, a plurality (e.g., two) of electrodes (e.g., the fourth electrode 114 and the third electrode 113 of FIG. 6A) may be used as a negative electrode.

As another example, in case that, as a result of analyzing the contact impedance difference from the electrocardiogram signal detected in the basic electrode connection state as shown in FIG. 4A, the contact impedance difference is greater than a configured value and the impedance of the positive electrode (e.g., the second electrode 112 of FIG. 4A) is greater than the impedance of the negative electrode (e.g., the fourth electrode 114 of FIG. 4A), the driving circuit 150 (or the processor 151) of the cover-type electronic device 100 may control the switching element 154_1 to change the electrode connection state as shown in FIG. 6B so as to compensate for the contact impedance. Accordingly, a plurality (e.g., two) of electrodes (e.g., the second electrode 112 and the first electrode 111 of FIG. 6A) may be used as a positive electrode.

In an embodiment, the switching operation for compensating for the contact impedance difference between both hands of the user may be performed together with the switching operation based on a user's gripping posture. For example, in a state in which the electrode connection state of FIG. 4A is switched to any one of the electrode connection states of FIGS. 4B, 4C, 4D, 4E, and 4F by a switching operation based on a user's gripping posture, at least one short-circuited electrode (e.g., the second electrode 112 of FIG. 4B) may be electrically connected to a positive electrode (e.g., the third electrode 113 of FIG. 4B) or a negative electrode (e.g., the first electrode 111 of FIG. 4B) such that the contact impedance difference between the two hands is compensated.

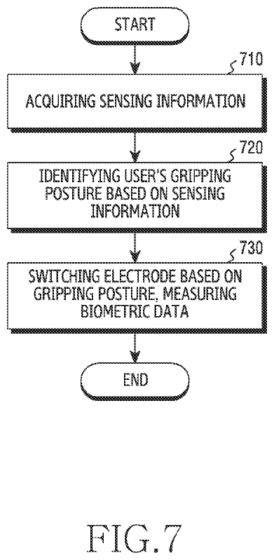

Hereinafter, a biometric data measuring method by a cover-type electronic device according to various embodiments will be described with reference to FIGS. 7, 8, 9, 10 and 11.

In various embodiments, some of the operations of the biometric data measuring method shown in FIGS. 7 to 11 may be omitted, the order of some operations may be changed, or other operations may be added. Alternatively, the operations of each embodiment may be selectively combined and then performed.

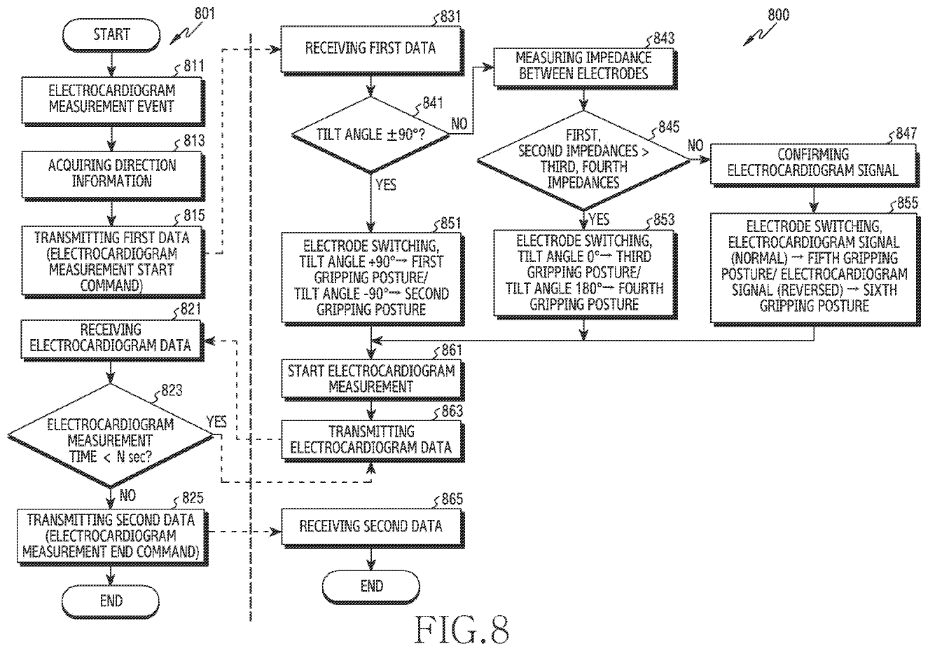

FIGS. 8, 10, and 11 exemplarily show methods for measuring biometric data by interaction between an electronic device (e.g., the electronic device 200) and a cover-type electronic device (e.g., the cover-type electronic device 100). In various embodiments, some operations are performed by the electronic device (e.g., the electronic device 200), and some operations are performed by the cover-type electronic device (e.g., the cover-type electronic device 100) (or the driving circuit (e.g., the driving circuit 150)) of the cover-type electronic device) or a processor (e.g., the processor 151). However, the subject performing each operation is not limited thereto, and may be variously changed, modified, applied, and extended according to embodiments. For example, some of the operations of the electronic device (e.g., the electronic device 200) may be performed by the cover-type electronic device (e.g., the cover-type electronic device 100) or some of the corresponding operations may be omitted.

FIG. 7 is a flowchart showing a biometric data measuring method by a cover-type electronic device according to an embodiment. For example, the method shown in FIG. 7 may correspond to an electrocardiogram data measuring method based on a gripping posture.

Referring to FIG. 7, a biometric data measuring method by a cover-type electronic device according to an embodiment may include operation 710, operation 720, and operation 730. For example, the biometric data measuring method shown in FIG. 7 may be performed by the cover-type electronic device 100 (or the driving circuit 150 or the processor 151 of the cover-type electronic device 100).

In operation 710, the cover-type electronic device 100 may acquire sensing information. For example, the sensing information may include one or more of direction information, information on impedance between a plurality of electrodes, and phase information of a user's biosignal (e.g., an electrocardiogram signal).

In operation 720, the cover-type electronic device 100 may identify a user's gripping posture (or the current gripping posture), which is one of a plurality of gripping postures for measuring designated biometric data (e.g., electrocardiogram data), based on the acquired sensing information. For example, the plurality of gripping postures may be gripping postures in which a user uses both hands. For example, the user's gripping posture may be identified as any one of the gripping postures shown in FIGS. 4A to 4F.

In operation 730, the cover-type electronic device 100 may switch a connection state of the electrode set 110, based on the identified gripping posture such that the designated biometric data is measured in the switched state. For example, biometric data may be measured after the connection state (or electrode connection state) of the electrode set 110 is switched to any one of the electrode connection states shown in FIGS. 4A to 4F.

FIG. 8 is a flowchart showing a biometric data measuring method using a cover-type electronic device and an electronic device according to an embodiment. For example, the method shown in FIG. 8 may correspond to an electrocardiogram data measuring method based on a gripping posture. For example, the method shown in FIG. 8 may be performed while the cover-type electronic device 100 is fastened to the electronic device 200 and/or short-range wireless communication between the cover-type electronic device 100 and the electronic device 200 is established. For example, reference numeral 800 may indicate operations performed by the cover-type electronic device 100. Reference numeral 801 may indicate operations performed by the electronic device 200.

In operation 811, the electronic device 200 may confirm the occurrence of an electrocardiogram measurement event. For example, in case that a designated application (e.g., a health application) is executed in the electronic device 200 or an electrocardiogram measurement menu in an application execution screen being displayed on a display of the electronic device 200 is executed (e.g., touch or tap for the menu) or in case that the electrode contact condition allowing electrocardiogram measurement is satisfied (e.g., a part of the user's body is in contact with three or more electrodes among the plurality of electrodes without a short circuit or a designated time has elapsed over the contacted state), the electronic device 200 may confirm the occurrence of an event for electrocardiogram measurement.

In operation 813, the electronic device 200 may acquire direction information of the electronic device 200 according to the occurrence for electrocardiogram measurement event. For example, the electronic device 200 may detect the direction of the electronic device 200 by using at least one internal sensor (e.g., an acceleration sensor, a gyro sensor, and/or an image sensor) thereof.

For example, the direction information of the electronic device 200 may include information on a tilt angle of the electronic device 200. The electronic device 200 may detect the tilt angle of the electronic device 200 by using at least one sensor (e.g., an acceleration sensor). For example, the tilt angle of the electronic device 200 may be any one of +90 degrees, −90 degrees, 0 degrees, and 180 degrees.

In operation 815, the electronic device 200 may transmit first data to the cover-type electronic device 100. For example, the first data may include direction information of the electronic device 200 and/or an electrocardiogram measurement start command.

In operation 831, the cover-type electronic device 100 may receive the first data transmitted from the electronic device 200. For example, the cover-type electronic device 100 may be switched from a sleep state to a normal state in response to receiving the first data including the electrocardiogram measurement start command.

Operations 841 to 855 may be operations of identifying a user's gripping posture and performing electrode switching based on the identified gripping posture.

For example, for a plurality of gripping postures, electrode information according to each gripping posture may be stored in advance, the user's current gripping posture may be identified, and an electrode switching operation may be performed in response to the electrode information according to the current gripping posture.

According to an embodiment, the cover-type electronic device 100 may identify the user's current gripping posture in response to information on the tilt angle of the electronic device 200.

According to an embodiment, the cover-type electronic device 100 may identify a user's current gripping posture in further consideration of information on impedance between electrodes and/or phase information of an electrocardiogram signal.

In operation 841, the cover-type electronic device 100 may determine whether the tilt angle of the electronic device 200 is ±90 degrees. For example, in case that the tilt angle of the electronic device 200 is ±90 degrees and in a predetermined offset range (e.g., within ±5 degrees), the tilt angle of the electronic device 200 may be determined as ±90 degrees.

In case that the tilt angle of the electronic device 200 is determined as ±90 degrees as the determination result in operation 841, the cover-type electronic device 100 may proceed to operation 851.

In operation 851, in case that the tilt angle of the electronic device 200 is +90 degrees, the cover-type electronic device 100 may determine the current gripping posture as the first gripping posture (e.g., the first gripping posture of FIG. 4A). For example, in case that the tilt angle of the electronic device 200 is +90 degrees, the cover-type electronic device 100 may determine that the current gripping posture is the first gripping posture, which is the basic gripping posture, and maintain the current electrode connection state without switching. As another example, the cover-type electronic device 100 may perform an electrode switching operation (e.g., switching to the corresponding electrode connection state in case that the current electrode connection state is not the electrode connection state (basic electrode connection state) as shown in FIG. 4A) to correspond to the first gripping posture.

In operation 851, in case that the tilt angle of the electronic device 200 is −90 degrees, the cover-type electronic device 100 may determine the current gripping posture as the second gripping posture (e.g., the second gripping posture of FIG. 4B). The cover-type electronic device 100 may perform an electrode switching operation (e.g., switching to the electrode connection state as shown in FIG. 4B) to correspond to the second gripping posture.

In case that the tilt angle of the electronic device 200 is not ±90 degrees as the determination result in operation 841, the cover-type electronic device 100 may proceed to operation 843.

In operation 843, the cover-type electronic device 100 may measure impedance between the electrodes. For example, the cover-type electronic device 100 may measure a first impedance between the first electrode 111 and the second electrode 112, a second impedance between the third electrode 113 and the fourth electrode 114, a third impedance between the first electrode 111 and the third electrode 113, and/or a fourth impedance between the second electrode 112 and the fourth electrode 114.

In operation 845, the cover-type electronic device 100 may compare impedances between electrodes, measured in operation 843, with each other. For example, the cover-type electronic device 100 may compare the first impedance between the first electrode 111 and the second electrode 112 and/or the second impedance between the third electrode 113 and the fourth electrode 114 with the third impedance between the first electrode 111 and the third electrode 113 and/or the fourth impedance between the second electrode 112 and the fourth electrode 114.

As a result of comparison in operation 845, in case that the first impedance and/or the second impedance are greater than the third impedance and/or the fourth impedance, the cover-type electronic device 100 may proceed to operation 853.

In operation 853, the cover-type electronic device 100 may determine the current gripping posture as the third gripping posture (e.g., the third gripping posture of FIG. 4C) or the fourth gripping posture (e.g., the fourth gripping posture of FIG. 4D), based on the tilt angle of the electronic device 200.

In operation 853, in case that the tilt angle of the electronic device 200 is 0 degrees, the cover-type electronic device 100 may determine the current gripping posture as the third gripping posture (e.g., the third gripping posture of FIG. 4C). For example, in case that the tilt angle of the electronic device 200 is 0 degrees and in a predetermined offset range (e.g., within ±5 degrees), the tilt angle of the electronic device 200 may be determined as 0 degrees. The cover-type electronic device 100 may perform an electrode switching operation (e.g., switching to the electrode connection state as shown in FIG. 4C) to correspond to the third gripping posture.

In operation 853, in case that the tilt of the electronic device 200 is 180 degrees, the cover-type electronic device 100 may determine the current gripping posture as the fourth gripping posture (e.g., the fourth gripping posture of FIG. 4D). For example, in case that the tilt angle of the electronic device 200 is 180 degrees and in a predetermined offset range (e.g., within ±5 degrees), the tilt angle of the electronic device 200 may be determined as 180 degrees. The cover-type electronic device 100 may perform an electrode switching operation (e.g., switching to the electrode connection state as shown in FIG. 4D) to correspond to the fourth gripping posture.

As a result of comparison in operation 845, in case that the first impedance and/or the second impedance are equal to or smaller than the third impedance and/or the fourth impedance, the cover-type electronic device 100 may proceed to operation 847.

In operation 847, the cover-type electronic device 100 may confirm an electrocardiogram signal measured through at least some of the plurality of electrodes.

In operation 855, in case that the electrocardiogram signal is in a normal state (or a normal phase), the cover-type electronic device 100 may determine the current gripping posture as the fifth gripping posture (e.g., the fifth gripping posture of FIG. 4E). For example, in case that the electrocardiogram signal is in a normal state, the current electrode connection state may be maintained (e.g., maintained in the electrode connection state as shown in FIG. 4E) without switching.

In operation 855, in case that the electrocardiogram signal is in a reversed state (or a reversed phase), the cover-type electronic device 100 may determine the current gripping posture as the sixth gripping posture (e.g., the sixth gripping posture of FIG. 4F). For example, in case that the electrocardiogram signal is in a reversed state, the cover-type electronic device 100 may perform an electrode switching operation (e.g., switching to the electrode connection state as shown in FIG. 4F) to correspond to the sixth gripping posture.

Through operations 841 to 855, the electrode connection state may be switched to correspond to a user's gripping posture.

In operation 861, the cover-type electronic device 100 may start electrocardiogram measurement through the electrode set 110 in a switched state. For example, the electrocardiogram measurement time may be counted from a time point at which the electrocardiogram measurement in operation 861 is started.

In operation 863, the cover-type electronic device 100 may continue the electrocardiogram measurement to transmit the measured electrocardiogram data to the electronic device 200.

In operation 821, the electronic device 200 may receive the electrocardiogram data from the cover-type electronic device 100.

In operation 823, the electronic device 200 may determine whether the electrocardiogram measurement time has reached a reference time (e.g., N sec, N=20-40) (or whether the electrocardiogram measurement time is smaller than the reference time).

In case that the electrocardiogram measurement time does not reach the reference time (or in case that the electrocardiogram measurement time is smaller than the reference time, operation 823—Yes), as the determination result in operation 823, the process may return to operation 863 and the electrocardiogram measurement may be continued through the cover-type electronic device 100.

As a result of the determination in operation 823, in case that the electrocardiogram measurement time reaches the reference time (or in case that the electrocardiogram measurement time is equal to or greater than the reference time, operation 823—NO), the electronic device 200 may proceed to operation 825.

In operation 825, the electronic device 200 may transmit second data to the cover-type electronic device 100. The second data may include an electrocardiogram measurement end command. The electronic device 200 may end the electrocardiogram measurement. For example, a message indicating that the electrocardiogram measurement has been completed and/or at least a part of the measured electrocardiogram data may be displayed on a screen of the electronic device 200 (e.g., a display of the electronic device 200), or the electrocardiogram data measured for a reference time may be stored (e.g., stored in a memory of the electronic device 200 or an operation server of a designated application (e.g., a health application)).

In operation 865, the cover-type electronic device 100 may receive the second data including the electrocardiogram measurement end command from the electronic device 200 and end the electrocardiogram measurement in response to the second data. For example, the cover-type electronic device 100 may return to the sleep state from the normal state in response to receiving the second data including the electrocardiogram measurement end command.

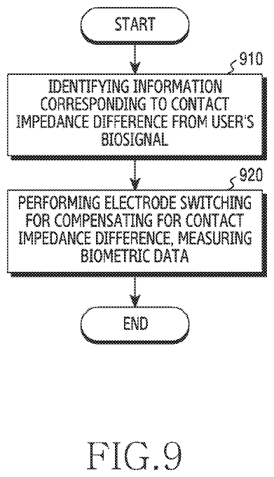

FIG. 9 is a flowchart showing a biometric data measuring method by a cover-type electronic device according to another embodiment. For example, the method shown in FIG. 9 may correspond to an electrocardiogram data measuring method for compensating for a contact impedance difference.

Referring to FIG. 9, the biometric data measuring method by a cover-type electronic device according to another embodiment may include operation 910 and operation 920. For example, the biometric data measuring method shown in FIG. 9 may be performed by the cover-type electronic device 100 (or the driving circuit 150 or the processor 151 of the cover-type electronic device 100).

In operation 910, the cover-type electronic device 100 may identify information corresponding to a contact impedance difference between both hands of a user from the biosignal detected through the electrode set 110. For example, the information may include a direct current offset of a biosignal.

The cover-type electronic device 100 may analyze the contact impedance difference between both hands of the user by using the information (e.g., a direct current offset) identified from the biosignal detected through the electrode set 110. For example, in case that the direct current offset of the biosignal is out of a normal level (e.g. −100 mV to 100 mV), the cover-type electronic device 100 may determine that the contact impedance difference between both hands of the user exceeds a configured value (e.g., a designated value among 10 to 100MΩ). In case that the direct current offset of the biosignal is out of the normal level, the biosignal level may be out of the measurable range (0˜1.2V) and measurement thereof may not be possible.

For example, in case that a connection state (or an electrode connection state) of the electrode set 110 and a user's gripping posture correspond to the state shown in FIG. 4A, according to the illustrated electrode connection state, the first electrode 111 may be connected to the ground terminal (RLD) and used as a ground electrode, the second electrode 112 may be connected to the positive terminal (INP) and used as a positive electrode, and the fourth electrode 114 may be connected to the negative terminal (INM) and used as a negative electrode. According to the illustrated user's gripping posture, one hand (e.g., the left hand) of the user may be in contact with the second electrode 112 serving as a positive electrode, and the other hand (e.g., the right hand) of the user may be in contact with the fourth electrode 114 serving as a negative electrode.

In this state, the cover-type electronic device 100 may analyze the contact impedance difference between both hands of the user through an electrocardiogram signal detected through the positive electrode (e.g., the second electrode 112 of FIG. 4A) and the negative electrode (e.g., the fourth electrode 114 of FIG. 4A).

For example, the cover-type electronic device 100 may detect a user's electrocardiogram signal for a designated reference time to extract a direct current component of the electrocardiogram signal or calculate a direct current offset corresponding to an average level of the electrocardiogram signal. The cover-type electronic device 100 may determine whether the contact impedance difference between both hands of the user exceeds a configured value, based on the direct current offset of the electrocardiogram signal. In case that the direct current offset of the electrocardiogram signal is out of the normal level, the cover-type electronic device 100 may determine that the contact impedance difference between both hands of the user exceeds the configured value. In case that the direct current offset of the electrocardiogram signal is within the normal level, the cover-type electronic device 100 may determine that the contact impedance difference between both hands of the user is equal to or smaller than the configured value.

In operation 920, the cover-type electronic device 100 may switch a connection state of the electrode set 110, based on information (e.g., a direct current offset of a biosignal) corresponding to a contact impedance difference between both hands of the user such that user's biometric data (e.g., electrocardiogram data) is measured in the switched state.

For example, the cover-type electronic device 100 may determine whether the contact impedance difference between both hands of the user is greater than the configured value, based on the direct current offset of the electrocardiogram signal after starting the electrocardiogram measurement and may perform switching in case that the contact impedance difference between both hands of the user is greater than the configured value.

In an embodiment, the electrode set 110 may include a positive electrode (e.g., the second electrode 112 of FIG. 4A), a negative electrode (e.g., the fourth electrode 114 of FIG. 4A), and at least one electrode (e.g., the third electrode 113 of FIG. 4A) in a short-circuited state. At least one electrode in a short-circuited state may be electrically connected to the positive electrode or the negative electrode by switching, and thus the cover-type electronic device 100 may compensate for a contact impedance difference between both hands of the user.

For example, by a switching operation for compensating for the contact impedance between both hands of the user, the connection state (or the electrode connection state) of the electrode set 110 may be changed from one of the states of FIGS. 4A to 4F to the state as shown in FIG. 6A or 6B.

For example, the cover-type electronic device 100 may connect two or more electrodes having a greater impedance to each other by switching to increase an electrode contact area, thereby compensating for the contact impedance to support normal electrocardiogram measurement.

For an example, in case that the impedance of an electrode on one side (e.g., one of the positive electrode and the negative electrode) is greater than the impedance of an electrode on the other side (e.g., the other one of the positive electrode and the negative electrode), the cover-type electronic device 100 may electrically connect at least one electrode in a short-circuited state to the electrode on one side. As another example, in case that the impedance of an electrode on the other side is greater than an electrode on one side, the cover-type electronic device 100 may electrically connect at least one electrode in a short-circuited state to the electrode on the other side.

In an embodiment, to increase the accuracy of the measurement, the cover-type electronic device 100 may detect a biosignal for a first reference time (e.g., 0.1 to 2 seconds) or longer to identify information corresponding to the contact impedance difference between both hands of the user from the biosignal and measure biometric data for a second reference time (e.g., 20 to 40 seconds) or longer to provide the same.

According to an embodiment, compensation for a contact impedance difference between the user's both hands by electrode switching may enable biometric data (e.g., electrocardiogram data) in the current user's body state without causing any inconvenience (e.g., moisturizing or exfoliating the skin for electrocardiography) to the user.

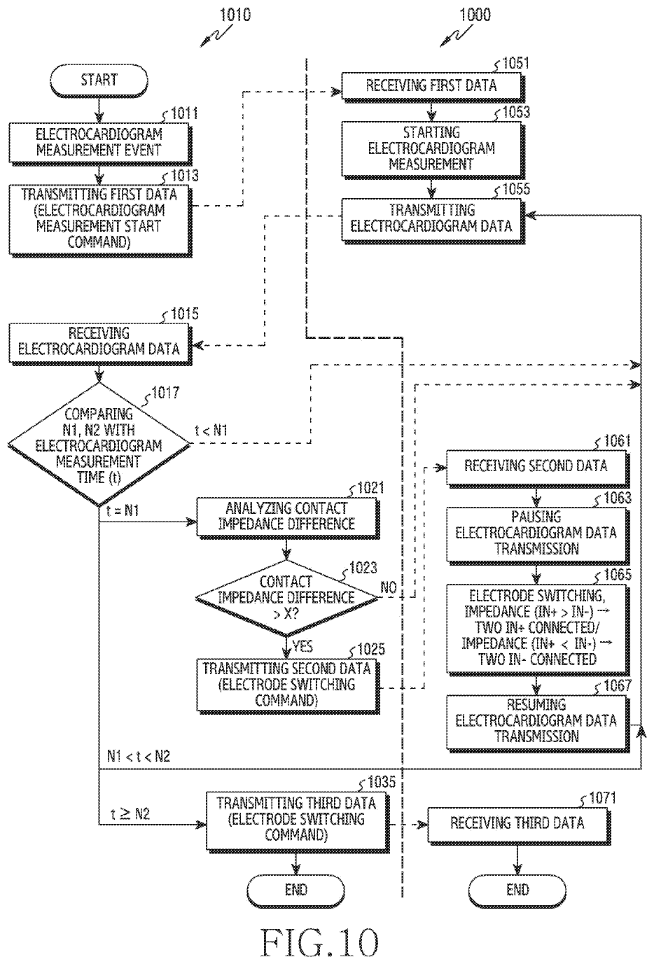

FIG. 10 is a flowchart showing a biometric data measuring method using a cover-type electronic device and an electronic device according to another embodiment. For example, the method illustrated in FIG. 10 may correspond to a method of measuring electrocardiogram data for compensating for contact impedance. For example, the method shown in FIG. 10 may be performed while the cover-type electronic device 100 is fastened to the electronic device 200 and/or short-range wireless communication between the cover-type electronic device 100 and the electronic device 200 is established. For example, reference numeral 1000 may indicate operations performed by the cover-type electronic device 100. Reference numeral 1010 may indicate operations performed by the electronic device 200.

In an embodiment of FIG. 10, the first reference time (e.g., N1 sec) may be an electrocardiogram measurement time for analyzing the contact impedance difference. The second reference time (e.g., N2 sec) may be an electrocardiogram measurement time for analyzing electrocardiogram.

In operation 1011, the electronic device 200 may confirm the occurrence of an electrocardiogram measurement event. For example, in case that a designated application (e.g., a health application) is executed in the electronic device 200 or an electrocardiogram measurement menu in an application execution screen being displayed on a display of the electronic device 200 is executed (e.g., touch or tap for the menu) or in case that a designated time has elapsed while a part of the user's body is in contact with at least one of the plurality of electrodes, the electronic device 200 may confirm the occurrence of an event for electrocardiogram measurement.

In operation 1013, the electronic device 200 may transmit first data to the cover-type electronic device 100. For example, the first data may include an electrocardiogram measurement start command.

In operation 1051, the cover-type electronic device 100 may receive the first data transmitted from the electronic device 200. For example, the cover-type electronic device 100 may switch from a sleep state to a normal state in response to receiving the first data including the electrocardiogram measurement start command.

In operation 1053, the cover-type electronic device 100 may start electrocardiogram measurement through the electrode set 110 including the plurality of electrodes. For example, an electrocardiogram measurement time (t) may be counted from a time point at which the electrocardiogram measurement in operation 1053 starts.

In operation 1055, the cover-type electronic device 100 may continue the electrocardiogram measurement to transmit the measured electrocardiogram data to the electronic device 200.

In operation 1015, the electronic device 200 may receive the electrocardiogram data from the cover-type electronic device 100.

In operation 1017, the electronic device 200 may compare the electrocardiogram measurement time (t) with the first reference time (e.g., N1 sec, N1=0.1-2) and/or the second reference time (e.g., N2 sec, N2=20-40). For example, the first reference time may have a value several times to several tens or more times greater than that of the second reference time.

As a result of comparison in operation 1017, in case that the electrocardiogram measurement time (t) is smaller than the first reference time (e.g., N1 sec) (in case that t<N1), the electronic device 200 may return to operation 1055 and thus the electrocardiogram measurement and the transmission of the measured electrocardiogram data may be continued through the cover-type electronic device 100.

As a result of comparison in operation 1017, in case that the electrocardiogram measurement time (t) reaches the first reference time (in case that t=N1), operation 1021 may be performed.

In operation 1021, the electronic device 200 may analyze a contact impedance difference. For example, the electronic device 200 may analyze the contact impedance difference, based on the electrocardiogram signal (or a direct current component of the electrocardiogram signal) detected through the electrode set 110 of the cover-type electronic device 100.

In operation 1023, the electronic device 200 may determine whether the contact impedance difference is greater than a configured value (e.g., X=10 MΩ).

In case that the contact impedance difference is equal to or smaller than the configured value (e.g., X) as the determination result in operation 1023, the electronic device 200 may return to operation 1055 and thus the electrocardiogram measurement may be continued through the cover-type electronic device 100.

In case that the contact impedance difference is greater than the configured value (e.g., X) as the determination result in operation 1023, operation 1025 may be performed. In operation 1025, the electronic device 200 may transmit second data to the cover-type electronic device 100. For example, the second data may include an electrode switching command.

In operation 1061, the cover-type electronic device 100 may receive the second data from the electronic device 200.

In operation 1063, the cover-type electronic device 100 may pause the electrocardiogram data transmission.

In operation 1065, the cover-type electronic device 100 may perform electrode switching. The cover-type electronic device 100 may perform an electrode switching operation to compensate for a contact impedance difference. For example, the cover-type electronic device 100 may analyze the contact impedance difference between both hands of the user from the electrocardiogram signal detected through the positive electrode and the negative electrode. In case that the contact impedance difference exceeds the configured value, the cover-type electronic device 100 may compare the impedance of the positive electrode and the impedance of the negative electrode with each other and connect at least one electrode in a short-circuited state to the electrodes having the greater larger impedance. For example, in case that the impedance of the positive electrode is greater than that of the negative electrode, the two positive electrodes may be connected to each other by switching, and thus the corresponding electrode contact area may be increased. As another example, in case that the impedance of the positive electrode is smaller than the impedance of the negative electrode, the two negative electrodes may be connected to each other by switching, and thus the corresponding electrode contact area may be increased.

In operation 1067, the cover-type electronic device 100 may resume electrocardiogram data transmission.

As a result of comparison in operation 1017, in case that the electrocardiogram measurement time (t) is greater than the first reference time (e.g., N1 sec) and smaller than the second reference time (e.g., N2 sec) (in case that N1<t<N2), the process may return to operation 1055 and thus electrocardiogram measurement and transmission of the measured electrocardiogram data may be continued.

As a result of comparison in operation 1017, in case that the electrocardiogram measurement time (t) is equal to or longer than the second reference time (e.g., N2 sec) (in case that t≥N2), operation 1035 may be performed.

In operation 1035, the electronic device 200 may transmit third data to the cover-type electronic device 100. The third data may include an electrocardiogram measurement end command. The electronic device 200 may end the electrocardiogram measurement.

In operation 1071, the cover-type electronic device 100 may receive the third data including the electrocardiogram measurement end command from the electronic device 200 and may terminate the electrocardiogram measurement in response to receiving the third data. For example, the cover-type electronic device 100 may return to the sleep state from the normal state in response to receiving the third data including the electrocardiogram measurement end command.

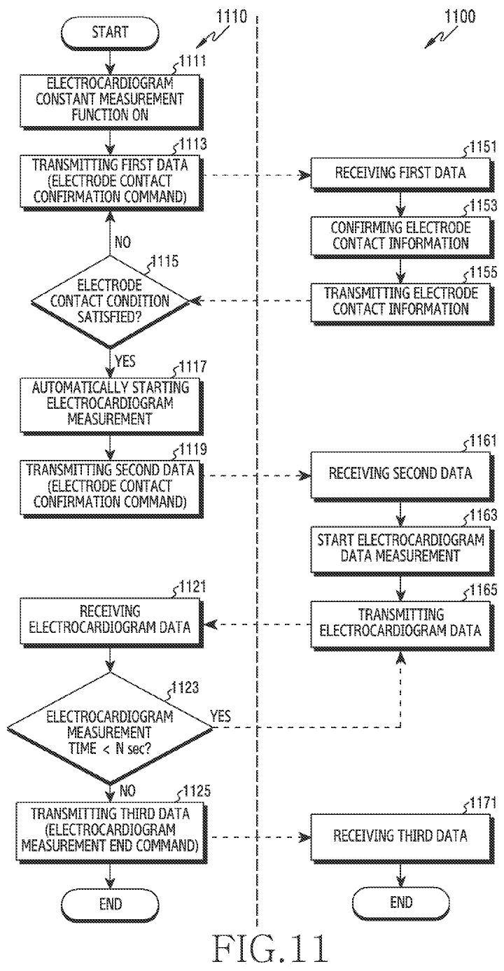

FIG. 11 is a flowchart showing a biometric data measuring method using a cover-type electronic device and an electronic device according to another embodiment. For example, the method illustrated in FIG. 11 may correspond to an electrocardiogram data measurement method supporting a quick start function. For example, reference numeral 1100 may indicate operations performed by the cover-type electronic device 100. Reference numeral 1110 may indicate operations performed by the electronic device 200.

In operation 1111, an electrocardiogram constant measurement function of the electronic device 200 may be configured to be in an on state. For example, by configuring the electrocardiogram measurement function to be in an on state, the electrocardiogram measurement may be automatically performed in response to gripping of electrodes by a user who needs to measure electrocardiogram frequently. In case that the electrocardiogram constant measurement function is configured to be in an on state, confirmation of whether the electrodes are in contact may be made all the time.

In operation 1113, the electronic device 200 may transmit first data to the cover-type electronic device 100. The first data may include an electrode contact confirmation command. For example, in case that the electrocardiogram constant measurement function is in an on state, the first data may be periodically transmitted to the cover-type electronic device 100 while the electronic device 200 is being in use (e.g., while the display of the electronic device 200 is turned on).

In operation 1151, the cover-type electronic device 100 may receive first data including an electrode contact confirmation command from the electronic device 200.

In operation 1153, the cover-type electronic device 100 may confirm electrode contact information. The electrode contact information may be information on whether each of the four electrodes (e.g., the first electrode 111, the second electrode 112, the third electrode 113, and the fourth electrode 114) in the electrode set 110 is in a contact state.

In operation 1155, the cover-type electronic device 100 may transmit the confirmed electrode contact information to the electronic device 200.

In operation 1115, the electronic device 200 may determine whether an electrode contact condition allowing electrocardiogram measurement is satisfied, based on the electrode contact information received from the cover-type electronic device 100. For example, the state in which a user is in contact with three or more electrodes (e.g., all four electrodes of the first electrode 111, the second electrode 112, the third electrode 113, and the fourth electrode 114 or three electrodes used as a positive electrode, a negative electrode, and a ground electrode among the four electrodes) may be determined that the electrode contact condition is satisfied. The case that the number of electrodes in contact with the user is less than three (e.g., all four electrodes are in a non-contact state) may be determined that the electrode contact condition is not satisfied. As another example, the case that a state in which the user is in contact with three or more electrodes is maintained for a designated time or longer may be determined that the electrode contact condition is satisfied, otherwise (e.g., the case that the contact state is released before the designated time or all four electrodes are in a non-contact state), the cover-type electronic device 100 may determine that the electrode contact condition is not satisfied.

In the case of the non-contact state as the determination result in operation 1115, the electronic device 200 may return to operation 1113 to confirm again whether the electrode is in contact state.

In the case of that the electrodes are in an electrode contact state as the determination result in operation 1115 (e.g., the case that a user is in contact with at least three of the four electrodes of the first electrode 111, the second electrode 112, the third electrode 113, and the fourth electrode 114), operation 1117 may proceed.

In operation 1117, the electronic device 200 may automatically start electrocardiogram measurement. For example, the electrocardiogram measurement time may be counted from a time point at which the electrocardiogram measurement in operation 1117 starts.

In operation 1119, the electronic device 200 may transmit second data to the cover-type electronic device 100. The second data may include an electrocardiogram measurement start command.

In operation 1161, the cover-type electronic device 100 may receive the second data including the electrocardiogram measurement start command from the electronic device 200.

In operation 1163, the cover-type electronic device 100 may start electrocardiogram data measurement.

In operation 1165, the cover-type electronic device 100 may transmit the measured electrocardiogram data to the electronic device 200.

In operation 1121, the electronic device 200 may receive electrocardiogram data from the cover-type electronic device 100.

In operation 1123, the electronic device 200 may determine whether the electrocardiogram measurement time has reached a reference time (e.g., N sec) (or whether the electrocardiogram measurement time is less than the reference time).

In case that the electrocardiogram measurement time does not reach the reference time (e.g., N sec) (or in case that the ECG measurement time is less than the reference time, operation 1123—Yes), as the determination result in operation 1123, the electronic device 200 may return to operation 1165 and the electrocardiogram measurement through the cover-type electronic device 100 may be continued.

In case that the electrocardiogram measurement time reaches the reference time (e.g., N sec) (or in case that the electrocardiogram measurement time is equal to or longer than the reference time, operation 1123—NO), as the determination result in operation 1123, operation 1125 may be performed.

In operation 1125, the electronic device 200 may transmit third data to the cover-type electronic device 100. The third data may include an electrocardiogram measurement end command.

In operation 1171, the cover-type electronic device 100 may receive the third data including the electrocardiogram measurement end command from the electronic device 200 and may end the electrocardiogram measurement in response thereto.

The embodiment of FIG. 11 (e.g., a quick start function) may be combined with the embodiments (e.g., an electrode switching operation or an electrocardiogram data measurement operation based on a user's gripping posture) of FIGS. 7 and 8 and/or the embodiments (e.g., an electrode switching operation for compensating for a contact impedance difference or an electrocardiogram data measurement operation) of FIGS. 9 and 10. As an example, the embodiment of FIG. 7 or 8 and the embodiment of FIG. 9 or 10 may support the quick start function described with reference to FIG. 11. As another example, the embodiment of FIG. 11 may further include an electrode switching operation based on a user's gripping posture described in FIGS. 7 and 8 or an electrode switching operation for compensating for a contact impedance difference described in FIGS. 9 and 10.

FIG. 12 is a block diagram illustrating an electronic device 1201 in a network environment 1200 according to various embodiments.

Referring to FIG. 12, the electronic device 1201 in the network environment 1200 may communicate with an electronic device 1202 via a first network 1298 (e.g., a short-range wireless communication network), or at least one of an electronic device 1204 or a server 1208 via a second network 1299 (e.g., a long-range wireless communication network). According to an embodiment, the electronic device 1201 may communicate with the electronic device 1204 via the server 1208. According to an embodiment, the electronic device 1201 may include a processor 1220, memory 1230, an input module 1250, a sound output module 1255, a display module 1260, an audio module 1270, a sensor module 1276, an interface 1277, a connecting terminal 1278, a haptic module 1279, a camera module 1280, a power management module 1288, a battery 1289, a communication module 1290, a subscriber identification module (SIM) 1296, or an antenna module 1297. In some embodiments, some of the components (e.g., the connecting terminal 1278) may be omitted from the electronic device 1201, or one or more other components may be added in the electronic device 1201. In some embodiments, some of the components (e.g., the sensor module 1276, the camera module 1280, or the antenna module 1297) may be implemented as a single component (e.g., the display module 1260).

The processor 1220 may execute, for example, software (e.g., a program 1240) to control at least one other component (e.g., a hardware or software component) of the electronic device 1201 coupled with the processor 1220, and may perform various data processing or computation. According to one embodiment, as at least part of the data processing or computation, the processor 1220 may store a command or data received from another component (e.g., the sensor module 1276 or the communication module 1290) in volatile memory 1232, process the command or the data stored in the volatile memory 1232, and store resulting data in non-volatile memory 1234. According to an embodiment, the processor 1220 may include a main processor 1221 (e.g., a central processing unit (CPU) or an application processor (AP)), or an auxiliary processor 1223 (e.g., a graphics processing unit (GPU), a neural processing unit (NPU), an image signal processor (ISP), a sensor hub processor, or a communication processor (CP)) that is operable independently from, or in conjunction with, the main processor 1221. For example, when the electronic device 1201 includes the main processor 1221 and the auxiliary processor 1223, the auxiliary processor 1223 may be adapted to consume less power than the main processor 1221, or to be specific to a specified function. The auxiliary processor 1223 may be implemented as separate from, or as part of the main processor 1221.

The auxiliary processor 1223 may control at least some of functions or states related to at least one component (e.g., the display module 1260, the sensor module 1276, or the communication module 1290) among the components of the electronic device 1201, instead of the main processor 1221 while the main processor 1221 is in an inactive (e.g., sleep) state, or together with the main processor 1221 while the main processor 1221 is in an active state (e.g., executing an application). According to an embodiment, the auxiliary processor 1223 (e.g., an image signal processor or a communication processor) may be implemented as part of another component (e.g., the camera module 1280 or the communication module 1290) functionally related to the auxiliary processor 1223. According to an embodiment, the auxiliary processor 1223 (e.g., the neural processing unit) may include a hardware structure specified for artificial intelligence model processing. An artificial intelligence model may be generated by machine learning. Such learning may be performed, e.g., by the electronic device 1201 where the artificial intelligence is performed or via a separate server (e.g., the server 1208). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 1230 may store various data used by at least one component (e.g., the processor 1220 or the sensor module 1276) of the electronic device 1201. The various data may include, for example, software (e.g., the program 1240) and input data or output data for a command related thereto. The memory 1230 may include the volatile memory 1232 or the non-volatile memory 1234.

The program 1240 may be stored in the memory 1230 as software, and may include, for example, an operating system (OS) 1242, middleware 1244, or an application 1246.

The input module 1250 may receive a command or data to be used by another component (e.g., the processor 1220) of the electronic device 1201, from the outside (e.g., a user) of the electronic device 1201. The input module 1250 may include, for example, a microphone, a mouse, a keyboard, a key (e.g., a button), or a digital pen (e.g., a stylus pen).

The sound output module 1255 may output sound signals to the outside of the electronic device 1201. The sound output module 1255 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 1260 may visually provide information to the outside (e.g., a user) of the electronic device 1201. The display module 1260 may include, for example, a display, a hologram device, or a projector and control circuitry to control a corresponding one of the display, hologram device, and projector. According to an embodiment, the display module 1260 may include a touch sensor adapted to detect a touch, or a pressure sensor adapted to measure the intensity of force incurred by the touch.

The audio module 1270 may convert a sound into an electrical signal and vice versa. According to an embodiment, the audio module 1270 may obtain the sound via the input module 1250, or output the sound via the sound output module 1255 or a headphone of an external electronic device (e.g., an electronic device 1202) directly (e.g., wiredly) or wirelessly coupled with the electronic device 1201.

The sensor module 1276 may detect an operational state (e.g., power or temperature) of the electronic device 1201 or an environmental state (e.g., a state of a user) external to the electronic device 1201, and then generate an electrical signal or data value corresponding to the detected state. According to an embodiment, the sensor module 1276 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1277 may support one or more specified protocols to be used for the electronic device 1201 to be coupled with the external electronic device (e.g., the electronic device 1202) directly (e.g., wiredly) or wirelessly. According to an embodiment, the interface 1277 may include, for example, a high definition multimedia interface (HDMI), a universal serial bus (USB) interface, a secure digital (SD) card interface, or an audio interface.

A connecting terminal 1278 may include a connector via which the electronic device 1201 may be physically connected with the external electronic device (e.g., the electronic device 1202). According to an embodiment, the connecting terminal 1278 may include, for example, a HDMI connector, a USB connector, a SD card connector, or an audio connector (e.g., a headphone connector).

The haptic module 1279 may convert an electrical signal into a mechanical stimulus (e.g., a vibration or a movement) or electrical stimulus which may be recognized by a user via his tactile sensation or kinesthetic sensation. According to an embodiment, the haptic module 1279 may include, for example, a motor, a piezoelectric element, or an electric stimulator.

The camera module 1280 may capture a still image or moving images. According to an embodiment, the camera module 1280 may include one or more lenses, image sensors, image signal processors, or flashes.

The power management module 1288 may manage power supplied to the electronic device 1201. According to one embodiment, the power management module 1288 may be implemented as at least part of, for example, a power management integrated circuit (PMIC).

The battery 1289 may supply power to at least one component of the electronic device 1201. According to an embodiment, the battery 1289 may include, for example, a primary cell which is not rechargeable, a secondary cell which is rechargeable, or a fuel cell.

The communication module 1290 may support establishing a direct (e.g., wired) communication channel or a wireless communication channel between the electronic device 1201 and the external electronic device (e.g., the electronic device 1202, the electronic device 1204, or the server 1208) and performing communication via the established communication channel. The communication module 1290 may include one or more communication processors that are operable independently from the processor 1220 (e.g., the application processor (AP)) and supports a direct (e.g., wired) communication or a wireless communication. According to an embodiment, the communication module 1290 may include a wireless communication module 1292 (e.g., a cellular communication module, a short-range wireless communication module, or a global navigation satellite system (GNSS) communication module) or a wired communication module 1294 (e.g., a local area network (LAN) communication module or a power line communication (PLC) module). A corresponding one of these communication modules may communicate with the external electronic device via the first network 1298 (e.g., a short-range communication network, such as Bluetooth™, wireless-fidelity (Wi-Fi) direct, or infrared data association (IrDA)) or the second network 1299 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 1292 may identify and authenticate the electronic device 1201 in a communication network, such as the first network 1298 or the second network 1299, using subscriber information (e.g., international mobile subscriber identity (IMSI)) stored in the subscriber identification module 1296.

The wireless communication module 1292 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 1292 may support a high-frequency band (e.g., the mmWave band) to achieve, e.g., a high data transmission rate. The wireless communication module 1292 may support various technologies for securing performance on a high-frequency band, such as, e.g., beamforming, massive multiple-input and multiple-output (massive MIMO), full dimensional MIMO (FD-M IMO), array antenna, analog beam-forming, or large scale antenna. The wireless communication module 1292 may support various requirements specified in the electronic device 1201, an external electronic device (e.g., the electronic device 1204), or a network system (e.g., the second network 1299). According to an embodiment, the wireless communication module 1292 may support a peak data rate (e.g., 20 Gbps or more) for implementing eMBB, loss coverage (e.g., 1264 dB or less) for implementing mMTC, or U-plane latency (e.g., 0.5 ms or less for each of downlink (DL) and uplink (UL), or a round trip of 12 ms or less) for implementing URLLC.

The antenna module 1297 may transmit or receive a signal or power to or from the outside (e.g., the external electronic device) of the electronic device 1201. According to an embodiment, the antenna module 1297 may include an antenna including a radiating element composed of a conductive material or a conductive pattern formed in or on a substrate (e.g., a printed circuit board (PCB)). According to an embodiment, the antenna module 1297 may include a plurality of antennas (e.g., array antennas). In such a case, at least one antenna appropriate for a communication scheme used in the communication network, such as the first network 1298 or the second network 1299, may be selected, for example, by the communication module 1290 (e.g., the wireless communication module 1292) from the plurality of antennas. The signal or the power may then be transmitted or received between the communication module 1290 and the external electronic device via the selected at least one antenna. According to an embodiment, another component (e.g., a radio frequency integrated circuit (RFIC)) other than the radiating element may be additionally formed as part of the antenna module 1297.

According to various embodiments, the antenna module 1297 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 1201 and the external electronic device 1204 via the server 1208 coupled with the second network 1299. Each of the electronic devices 1202 or 1204 may be a device of a same type as, or a different type, from the electronic device 1201. According to an embodiment, all or some of operations to be executed at the electronic device 1201 may be executed at one or more of the external electronic devices 1202, 1204, or 1208. For example, if the electronic device 1201 should perform a function or a service automatically, or in response to a request from a user or another device, the electronic device 1201, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 1201. The electronic device 1201 may provide the outcome, with or without further processing of the outcome, as at least part of a reply to the request. To that end, a cloud computing, distributed computing, mobile edge computing (MEC), or client-server computing technology may be used, for example. The electronic device 1201 may provide ultra low-latency services using, e.g., distributed computing or mobile edge computing. In another embodiment, the external electronic device 1204 may include an internet-of-things (IoT) device. The server 1208 may be an intelligent server using machine learning and/or a neural network. According to an embodiment, the external electronic device 1204 or the server 1208 may be included in the second network 1299. The electronic device 1201 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 1240) including one or more instructions that are stored in a storage medium (e.g., internal memory 1236 or external memory 1238) that is readable by a machine (e.g., the electronic device 1201). For example, a processor (e.g., the processor 1220) of the machine (e.g., the electronic device 1201) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as the functions are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

A cover-type electronic device according to various embodiments may include a cover-type housing (e.g., the housing 120 of FIG. 1), an electrode set (e.g., the electrode set 110 of FIG. 1) including a plurality of electrodes (e.g., the first electrode 111, the second electrode 112, the third electrode 113, and the fourth electrode 114 of FIG. 1) disposed outside the housing, and a printed circuit board (e.g., the printed circuit board 130 of FIG. 1) electrically connected to the plurality of electrodes. A driving circuit (e.g., the driving circuit 150 of FIG. 1) in the printed circuit board may be configured to acquire sensing information, identify a user's gripping posture, which is one of a plurality of gripping postures for measurement of designated biometric data, based on the sensing information, and switch a connection state of the electrode set, based on the gripping posture such that the designated biometric data is measured in the switched state.

According to various embodiments, the cover-type electronic device and an electronic device fastened to the housing may be connected via short-range wireless communication through the driving circuit.

According to various embodiments, the sensing information may include direction information, and the direction information may be received from the electronic device via the short-range wireless communication.

According to various embodiments, the driving circuit may be configured to transmit the measured biometric data to the electronic device via the short-range wireless communication.

According to various embodiments, the sensing information may include at least one of information on impedance between the plurality of electrodes and phase information of a user's biosignal.

According to various embodiments, the driving circuit may be configured to include a communication circuit for short-range wireless communication, a biosensor configured to measure the designated biometric data, a switching circuit disposed between the plurality of electrodes and the biosensor, a power control circuit configured to receive power supplied from the electronic device fastened to the housing, to drive the cover-type electronic device, and at least one processor electrically connected to at least one of the communication circuit, the biosensor, the switching circuit, and the power control circuit.

According to various embodiments, the plurality of electrodes may be disposed to correspond to corners of the housing, respectively.

According to various embodiments, entry into a measurement mode may be made in response to a contact being detected for a configured time or longer with respect to the plurality of electrodes.

According to various embodiments, entry into a measurement mode may be made in response to an event for starting measurement of the designated biometric data, occurring in the electronic device. The measurement mode may be terminated in response to an event for terminating measurement of the designated biometric data, occurring in the electronic device.

According to various embodiments, the plurality of electrodes may include four electrodes. Two electrodes may be disposed on one side of the housing to be spaced apart from each other. Two electrodes may be disposed on the other side of the housing to be spaced apart from each other.

According to various embodiments, the designated biometric data may be electrocardiogram data. The plurality of gripping postures may be gripping postures in which a user uses both hands.

According to various embodiments, the driving circuit may be configured to identify information corresponding to a contact impedance difference between the user's both hands from a biosignal detected through the electrode set, and switch a connection state of the electrode set, based on information corresponding to the contact impedance difference between both hands of the user.

According to various embodiments, at least some of the positive electrode, the negative electrode, and the ground electrode in the electrode set may be changed by switching based on the gripping posture. At least one electrode in the electrode set may be electrically connected to the positive electrode or the negative electrode such that a contact impedance difference between the user's both hands is compensated.

A cover-type electronic device according to various embodiments may include a cover-type housing (e.g., the housing 120 of FIG. 1), an electrode set (e.g., the electrode set 110 of FIG. 1) including a plurality of electrodes (e.g., the first electrode 111, the second electrode 112, the third electrode 113, and the fourth electrode 114 of FIG. 1) disposed outside the housing, and a printed circuit board (e.g., the printed circuit board 130 of FIG. 1) electrically connected to the plurality of electrodes. A driving circuit (e.g., the driving circuit 150 of FIG. 1) in the printed circuit board may be configured to identify information corresponding to a contact impedance difference between both hands of the user from a biosignal detected through the electrode set, and switch a connection state of the electrode set, based on the information corresponding to the contact impedance difference between both hands of the user such that the user's biometric data is measured in the switched state.

According to various embodiments, the electrode set may include a positive electrode, a negative electrode, and at least one electrode in a short-circuited state. The at least one electrode may be electrically connected to the positive electrode or the negative electrode by the switching such that a contact impedance difference between the user's both hands is compensated.

According to various embodiments, the driving circuit may be configured to perform the switching in case that a contact impedance difference between the user's both hands is greater than a configured value.

According to various embodiments, in case that the impedance of an electrode on one side, which is one of the positive electrode and the negative electrode, is greater than the impedance of an electrode on the other side, which is the other one of the positive electrode and the negative electrode, the at least one electrode may be electrically connected to the electrode on one side. In case that the impedance of the electrode on the other side is greater than the impedance of the electrode on one side, the at least one electrode may be electrically connected to the electrode on the other side.

According to various embodiments, the driving circuit may be configured to detect the biosignal for a first reference time or longer, and measure the biometric data for a second reference time or longer.

A biometric data measuring method by a cover-type electronic device including an electrode set according to various embodiments may include acquiring sensing information, identifying one of user's gripping postures for measurement of designated biometric data, based on the sensing information, and switching the electrode set, based on the gripping posture such that designated biometric data is measured in the switched state.

The biometric data measuring method by a cover-type electronic device including an electrode set according to various embodiments may include identifying information corresponding to a contact impedance difference between both hands of the user by using a biosignal detected through the electrode set, and switching a connection state of the electrode set, based on the information corresponding to the contact impedance difference between both hands of the user such that the user's biometric data is measured in the switched state.

Although the present disclosure has been described with various embodiments, various changes and modifications may be suggested to one skilled in the art. It is intended that the present disclosure encompass such changes and modifications as fall within the scope of the appended claims.

What is claimed is:

1. A cover-type electronic device comprising:
a cover-type housing;
an electrode set comprising a plurality of electrodes disposed on an exterior surface of the housing; and
a printed circuit board electrically connected to the plurality of electrodes,
wherein a driving circuit in the printed circuit board is configured to:
acquire sensing information;
identify a gripping posture of a user that matches one of a plurality of gripping postures for measurement of designated biometric data, based on the sensing information; and
switch a connection state of the electrode set from a first state to a switched state, based on the identified gripping posture such that the designated biometric data is measured in the switched state.

2. The cover-type electronic device of claim 1, wherein the housing is configured to fasten to an electronic device; and
wherein the driving circuit is further configured to connect the cover-type electronic device to the electronic device fastened to the housing via short-range wireless communication.

3. The cover-type electronic device of claim 2, wherein the sensing information comprises direction information, and
the driving circuit is further configured to receive the direction information from the electronic device via the short-range wireless communication.

4. The cover-type electronic device of claim 2, wherein the driving circuit is configured to transmit the measured biometric data to the electronic device via the short-range wireless communication.

5. The cover-type electronic device of claim 1, wherein the sensing information comprises at least one of phase information of a biosignal of the user or information on impedance between the plurality of electrodes.

6. The cover-type electronic device of claim 1, wherein the driving circuit comprises:
a communication circuit for short-range wireless communication;
a biosensor configured to measure the designated biometric data;
a switching circuit disposed between the plurality of electrodes and the biosensor;
a power control circuit configured to receive power supplied from the electronic device fastened to the housing and to drive the cover-type electronic device using the received power; and
at least one processor electrically connected to at least one of the communication circuit, the biosensor, the switching circuit, or the power control circuit.

7. The cover-type electronic device of claim 1, wherein the plurality of electrodes are disposed to correspond to corners of the housing, respectively.

8. The cover-type electronic device of claim 1, wherein the cover-type electronic device is configured to enter into a measurement mode in response to detecting a contact for a configured time or longer with respect to the plurality of electrodes.

9. The cover-type electronic device of claim 2, wherein the cover-type electronic device is configured to:
enter into a measurement mode in response to an event for starting measurement of the designated biometric data, the event for starting measurement occurring in the electronic device; and
terminate the measurement mode in response to an event for terminating measurement of the designated biometric data, the event for terminating measurement occurring in the electronic device.

10. The cover-type electronic device of claim 1, wherein the plurality of electrodes comprise four electrodes, including
two electrodes that are disposed on one side of the housing and spaced apart from each other, and
two electrodes that are disposed on another side of the housing and spaced apart from each other.

11. The cover-type electronic device of claim 1, wherein the designated biometric data is electrocardiogram data, and
the plurality of gripping postures are gripping postures in which the user uses both hands.

12. The cover-type electronic device of claim 1, wherein the driving circuit is configured to:
identify information corresponding to a contact impedance difference between both hands of the user from a biosignal detected through the electrode set; and
switch the connection state of the electrode set, based on information corresponding to the contact impedance difference between both hands of the user.

13. The cover-type electronic device of claim 1, wherein:
the switching of the connection state of the electrode set based on the identified gripping posture changes at least specific electrodes from among a positive electrode, a negative electrode, and a ground electrode in the electrode set; and
at least one electrode in the electrode set is electrically connected to the positive electrode or the negative electrode such that a contact impedance difference between both hands of the user is compensated.

14. A cover-type electronic device comprising:
a cover-type housing;
an electrode set comprising a plurality of electrodes disposed on an exterior surface of the housing; and
a printed circuit board electrically connected to the plurality of electrodes,
wherein a driving circuit in the printed circuit board is configured to:
identify information corresponding to a contact impedance difference between both hands of a user from a biosignal detected through the electrode set; and
switch a connection state of the electrode set from a first state to a switched state, based on the information corresponding to the contact impedance difference between both hands of the user such that biometric data of the user is measured in the switched state.

15. The cover-type electronic device of claim 14, wherein:
the electrode set comprises a positive electrode, a negative electrode, and at least one electrode in a short-circuited state; and
the switching of the connection state of the electrode set electrically connects the at least one electrode to the positive electrode or the negative electrode such that the contact impedance difference between both hands of the user is compensated.

16. The cover-type electronic device of claim 14, wherein the driving circuit is configured to perform the switching when the contact impedance difference between both hands of the user is greater than a configured value.

17. The cover-type electronic device of claim 15, wherein:
a first electrode is one of the positive electrode and the negative electrode, and a second electrode is the other one of the positive electrode and the negative electrode;
when an impedance of the first electrode on one side of the housing is greater than an impedance of the second electrode on another side of the housing, the at least one electrode is electrically connected to the first electrode on the one side; and
when the impedance of the second electrode on the other side is greater than the impedance of the first electrode on the one side, the at least one electrode is electrically connected to the second electrode on the other side.

18. The cover-type electronic device of claim 14, wherein the driving circuit is configured to:
detect the biosignal for a first reference time or longer; and
measure the biometric data for a second reference time or longer.

19. A biometric data measuring method performed by a cover-type electronic device that comprises an electrode set, the method comprising:
acquiring sensing information;
identifying a gripping posture of a user that matches one of a plurality of gripping postures for measurement of designated biometric data, based on the sensing information; and
switching the electrode set from a first state to a switched state, based on the identified gripping posture such that the designated biometric data is measured in the switched state.

20. The biometric data measuring method of claim 19, wherein:
the acquiring of the sensing information comprises acquiring the sensing information from a sensor of an electronic device that is fastened to a housing of the cover-type electronic device; and
the method further comprises after switching the electrode set to the switched state, measuring, by a biosensor of the cover-type electronic device, the designated biometric data.
